SEQUENCE LISTING (48 sequences)

SEQ ID NO: 1 - protein, 17 aa, artificial sequence
  antibody fragment
KSSQSLLNSSNQKNYLA

SEQ ID NO: 2 - protein, 7 aa, artificial sequence
  antibody fragment
FASTRDS

SEQ ID NO: 3 - protein, 9 aa, artificial sequence
  antibody fragment
QQHYILPPT

SEQ ID NO: 4 - protein, 14 aa, artificial sequence
  antibody fragment
RSSTGAVTTSNYAN

SEQ ID NO: 5 - protein, 7 aa, artificial sequence
  antibody fragment
GTNNRAP

SEQ ID NO: 6 - protein, 9 aa, artificial sequence
  antibody fragment
ALWYSNLWV

SEQ ID NO: 7 - protein, 11 aa, artificial sequence
  antibody fragment
RASENIFIYLA

SEQ ID NO: 8 - protein, 7 aa, artificial sequence
  antibody fragment
NAKTLAE

SEQ ID NO: 9 - protein, 9 aa, artificial sequence
  antibody fragment
QHHYGIPFT

SEQ ID NO: 10 - protein, 5 aa, artificial sequence
  antibody fragment
EYTMH

SEQ ID NO: 11 - protein, 17 aa, artificial sequence
  antibody fragment
SINPNNGGTNYNQKFKG

SEQ ID NO: 12 - protein, 8 aa, artificial sequence
  antibody fragment
TLYYAMDY

SEQ ID NO: 13 - protein, 5 aa, artificial sequence
  antibody fragment
SYGVH

SEQ ID NO: 14 - protein, 16 aa, artificial sequence
  antibody fragment
VIWAGGVTHYNSALMS

SEQ ID NO: 15 - protein, 11 aa, artificial sequence
  antibody fragment
GGALLRSHFDY

SEQ ID NO: 16 - protein, 5 aa, artificial sequence
  antibody fragment
DTYIH

SEQ ID NO: 17 - protein, 17 aa, artificial sequence
  antibody fragment
RIDPANGNTKYDPKLQG

SEQ ID NO: 18 - protein, 12 aa, artificial sequence
  antibody fragment
RDGTYVYYAMDY

SEQ ID NO: 19 - protein, 113 aa, artificial sequence
  antibody fragment
DIVMTQSPSSLAMSVGQKVTMSCKSSQSLLNSSNQKNYLAWYQQKPRQSPKLLVYFASTRDSGVPDRFLGSGSGTDFTLTINRVQAEDLADYFCQQHYILPPTFGAGTKLELK

SEQ ID NO: 20 - protein, 109 aa, artificial sequence
  antibody fragment
QAVVTQESALTTSPGETVTLTCRSSTGAVTTSNYANWVQEKPDHLFTGLIGGTNNRAPGVPARFSGSLIGDKAALTITGAQTEDEAIYHCALWYSNLWVFGGRTKLTVL

SEQ ID NO: 21 - protein, 107 aa, artificial sequence
  antibody fragment
DIQMTQSPASLSASVGETVTITCRASENIFIYLAWYQQKQGKSPQLLVYNAKTLAEGVPSRFSGSGSGTQFSLKINSLLPEDFGSYYCQHHYGIPFTFGSGTKLEIE

SEQ ID NO: 22 - protein, 117 aa, artificial sequence
  antibody fragment
EVQLQQSGPELVKPGASVKISCKTSGYTFTEYTMHWVKQSHGKSLEWIGSINPNNGGTNYNQKFKGKATLTVDKSSNTAYMELRSLTSGDSAVYYCARTLYYAMDYWGQGTSVTVSS

SEQ ID NO: 23 - protein, 119 aa, artificial sequence
  antibody fragment
QVQLKESGPGLVAPSQSLSITCTVSGFSLTSYGVHWVRQPPGKGLEWLGVIWAGGVTHYNSALMSRLNISKDNSQSQVFLKMNSLRTDDTAMYYCARGGALLRSHFDYWGQGTTLTVSS

SEQ ID NO: 24 - protein, 120 aa, artificial sequence
  antibody fragment
EVQLQQSGAELVKPGASVKLSCTVSGFNITDTYIHWVKQRPEKGLEWIGRIDPANGNTKYDPKLQGKATITADTSSNTAYLQLSSLTSEDTAVYYCARDGTYVYYAMDYWGQGTSVTVSS

SEQ ID NO: 25 - protein, 152 aa, artificial sequence
  recombinant IL1RA
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 26 - protein, 177 aa, artificial sequence
  recombinant IL1RA
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 27 - DNA, 399 bp, artificial sequence
  antibody fragment
atggaatcacagacccaggtcctcatgtttcttctgctctgggtatctggtgcctgtgcagacattgtgatgacacagtctccatcctccctggctatgtcagtaggacagaaggtcactatgagctgcaagtccagtcagagccttttaaatagtagtaatcaaaagaactatttggcctggtaccagcagaaaccaagacagtctcctaaacttctggtatactttgcatccactagggattctggggtccctgatcgcttcttaggcagtggatctgggacagatttcactcttaccatcaaccgtgtgcaggctgaagacctggcagattacttctgtcagcaacattatattcttcctcccacgttcggtgctgggaccaagctggagctgaaa

SEQ ID NO: 28 - DNA, 408 bp, artificial sequence
  antibody fragment
atgggatggagctggatctttctctttctcctgtcaggaactgcaggtgtcctctctgaggtccagctgcaacagtctggacctgagctggtgaagcctggggcttcagtgaagatatcctgcaagacttctggatacacattcactgaatacaccatgcactgggtgaagcagagccatggaaagagccttgagtggattggaagtattaatcctaacaatggtggtactaactacaaccagaagttcaagggcaaggccacattgactgtagacaagtcctccaacacagcctacatggagctccgcagcctgacatctggtgattctgcagtctattactgtgcaagaactctctactatgctatggactactggggtcaaggaacctcagtcaccgtctcctca

SEQ ID NO: 29 - DNA, 384 bp, artificial sequence
  antibody fragment
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatcactgtgctctatggtacagcaacctttgggtgttcggtggaagaaccaaactgactgtccta

SEQ ID NO: 30 - DNA, 414 bp, artificial sequence
  antibody fragment
atggctgtcctggtgctgttcctctgcctggttgcatttccaagctgtgtcctgtcccaggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggagtcacacattataattcggctctcatgtccagactgaacatcagcaaagacaactcccagagccaagttttcttaaaaatgaacagtctacgaactgatgacacagccatgtactactgtgccagaggtggggcattacttcggtctcactttgactactggggccaaggcaccactctcacagtctcctca

SEQ ID NO: 31 - DNA, 381 bp, artificial sequence
  antibody fragment
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatattttcatttatttagcatggtatcagcaaaaacagggaaaatctcctcagctcctggtctataatgcaaagaccttagcagaaggtgtgccatcaaggttcagtggcagtggatcaggcacacagttttctctgaagatcaacagccttctgcctgaagattttgggagttattactgtcaacatcattatggtattccattcacgttcggctcggggacaaagttggaaatagaa

SEQ ID NO: 32 - DNA, 417 bp, artificial sequence
  antibody fragment
atgaaatgcagctgggttatcttcttcctgatggcagtggttacaggggtcaattcagaggttcagctgcagcagtctggggcagagcttgtgaagccaggggcctcagtcaagttgtcctgcacagtttctggtttcaacattacagacacctatatacactgggtgaagcagaggcctgaaaagggcttggagtggattggaaggattgatcctgcgaatggtaatactaaatatgacccgaagttacagggcaaggccactataacagcagacacatcctccaacacagcctacctgcagctcagcagcctgacatctgaggacactgccgtctattactgtgctagagatggtacctacgtttactatgctatggactactggggtcaaggaacctcagtcaccgtctcctca

SEQ ID NO: 33 - protein, 159 aa, Artificial Sequence
  isoforme
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 34 - protein, 180 aa, Artificial Sequence
  isoforme
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 35 - protein, 143 aa, Artificial Sequence
  isoforme
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 36 - protein, 10 aa, Artificial Sequence
  epitope
FRIWDVNQKT

SEQ ID NO: 37 - protein, 10 aa, Artificial Sequence
  epitope
GYLQGPNVNL

SEQ ID NO: 38 - protein, 6 aa, Artificial Sequence
  epitope
DSGPTT

SEQ ID NO: 39 - protein, 6 aa, Artificial Sequence
  epitope
AMEADQ

SEQ ID NO: 40 - protein, 17 aa, Artificial Sequence
  epitope
NMPDEGVMVTKFYFQED

SEQ ID NO: 41 - protein, 8 aa, Artificial Sequence
  epitope
FYLRNNQL

SEQ ID NO: 42 - protein, 10 aa, Artificial Sequence
  epitope
LQGPVNLEEK

SEQ ID NO: 43 - protein, 7 aa, Artificial Sequence
  epitope
HGGGMCL

SEQ ID NO: 44 - DNA, 384 bp, Artificial Sequence
  antibody VL
atggattttcaagtgcagattttcagcttcctgctaatgagtgcctcagtcataatgtccaggggacaaattgttctcacccagtctccagcactcatgtctgcatctccaggggagaaggtcaccatgacctgcagtgccagctcaagtgtaagttacatgtactggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggagtagtaacccactcacgttcggtgctgggaccaagctggagctgaaa

SEQ ID NO: 45 - DNA, 418 bp, Artificial Sequence
  antibody VH
atgatggtgttaagtcttctgtacctgttgacagcccttccgggtatcctgtcagacgtgcagcttcaggatcaggacctagcctcgtgaaaccttctcagactctgtccctcacctgttctgtcactggcgactccatcacagtggttactggaactggatccggaaattcccagggaataaacttgagtacatggggtacataagctacgtggtagcacttactacaatccatctctcaaaagtcgaatctccatcactcgagacacatccaagaaccatactacctgcagttgaattctgtgactactgaggacacagccacatattactgtgcaagatgggggcatgttactacggtagtagctactggtacttcgatgtctggggcgcagggaccacggtcaccgtctcctca

SEQ ID NO: 46 - DNA, 381 bp, Artificial Sequence
  antibody VL
atggtatccacacctcagttccttgtatttttgcttttctggattccagcctccagaagtgacatcttgctgactcagtctccagccatcctgtctgtgagtccaggagacagagtcagtttctcctgcagggccagtcagaccattggcacaaacatacactggtatcaccaaacaacaaatggttctccaaggcttctcattaagtatgcttctgagtctatctctgggatcccttccaggtttagtggcagtggatcagggacagattttactcttaccatcatcagtgtggagtctgaagatattgcagattattactgtcaacaaagtaatagctggccgctcacgttcggtgctgggaccaagctggagctgaaa

SEQ ID NO: 47 - DNA, 420 bp, Artificial Sequence
  antibody VH
atgggatggagctgtatcatcctcttcttggtagcaacagctacaggtgtccactcccaggtccaactgcagcagcctggggctgagctggtgaggcctggggcttcagtgaacctgtcctgcaaggcttctggctacaccttcaccaactactggataaactgggtgaagcagaggcctggacaaggccttgagtggatcggaaatattcatccttatgatagttatactcactacaatcaaaagttcaagggcaaggccacattgactgtagacaaatcctccagcacggcctacatgcagctcagcagcccgacatctgaggactctgcagtctatttctgtacaaggaggggggtacgacgggacgactactactttgactactggggccaaggcaccactctcacagtctcctca

SEQ ID NO: 48 - protein, 151 aa, Artificial Sequence
  truncated protein
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQED

(19) United States
(12) Patent Application Publication
(10) Pub. No.: US 2023/0167179 A1
(43) Pub. Date: Jun. 1, 2023

(54) ANTIBODY AND USES THEREOF

(71) Applicants: CENTRE NATIONAL DE LA RECHERCHE SCIENTIFIQUE, PARIS (FR); UNIVERSITÉ DE RENNES 1, RENNES CEDEX (FR)

(72) Inventors: Hugues GASCAN, Angers (FR); Sylvie CHEVALIER, Angers (FR)

(73) Assignees: CENTRE NATIONAL DE LA RECHERCHE SCIENTIFIQUE, PARIS (FR); UNIVERSITÉ DE RENNES 1, RENNES CEDEX (FR)

(21) Appl. No.: 17/921,792

(22) PCT Filed: Apr. 30, 2021
(86) PCT No.: PCT/EP2021/061451

§ 371 Date: Oct. 27, 2022

(30) Foreign Application Priority Data
Apr. 30, 2020 (FR) 2004349

(51) Int. Cl. C07K 16/28; G01N 33/68; A61P 31/04

(52) U.S. Cl. CPC C07K 16/2866; G01N 33/6869; A61P 31/04; C07K 2317/76; C07K 2317/92; G01N 2333/7155; G01N 2333/20; A61K 2039/505

(57) ABSTRACT

An isolated monoclonal antibody against the interleukin 1-alpha or beta receptor antagonist, known as IL-1RA. The antibody inhibits the interaction of IL-1RA with the membrane-binding chain thereof, the interleukin-1 receptor. Also, nucleic acid molecule encoding for a light chain of the monoclonal antibody, and a nucleic acid molecule encoding for a heavy chain of the monoclonal antibody. Also, a method for treating an inflammatory, infectious or autoimmune disease, which includes administering the isolated monoclonal antibody or a nucleic acid coding for the isolated monoclonal antibody, and a method of or the diagnosis of an inflammatory, infectious or autoimmune diseases, which includes contacting a biological sample with the isolated monoclonal antibody.

### FIELD

The invention relates to an antibody and its uses, in particular in the context of treating inflammatory, infectious or autoimmune diseases.

### BACKGROUND

Since its description in the early 1980s, Lyme disease, caused by a family of bacteria, the Borrelia transmitted by tick bites, has evolved into a major epidemic to become the first zoonosis in the northern hemisphere. In 2018, the declared number of new cases in France approached 70,000, which is as much as the number of breast cancers, and 10 times more than the annual new cases of AIDS. But this is a minimum count, only counting the acute forms of the pathology, easily diagnosed by the characteristic presence of an inflammatory ring in the skin generally centered on the bite.

However, there is also a late, or chronic, disseminated form of the disease that is characterized by polyarthralgia, “desocializing” chronic fatigue, major cognitive impairment, sometimes also with significant motor or cardiac impairment. The official definition of this complex form of the disease by the French High Authority for Health [Haute Autorité de Santé française] is recent (June 2018), and was cross-validated by a decision of the Council of State of December 2019.

There is currently no reliable biological test to support the diagnosis of the chronic form of the disease. This diagnosis is above all based on the clinical experience of the doctor for this particularly complex pathology.

The total count of the different forms is quite likely approaching 100,000 new cases in France per year. It is therefore an infectious pathology with an epidemic nature and for which, in its late form, there is currently no treatment that can treat patients with certainty, resulting in severe disabilities.

To date, there is therefore a real need to provide treatment for these patients suffering from the chronic form of the disease.

The invention aims to remedy the shortcoming of the prior art.

### SUMMARY

The invention relates to a monoclonal antibody, isolated from its natural context, against the interleukin-1, or IL-1RA, receptor, said antibody inhibiting the interaction between the interleukin-1 receptor antagonist and the interleukin 1 receptor, said antibody having an affinity for IL-1RA of less than 10⁻⁸ M, in particular of the order of 10⁻⁹ M, this affinity being measured by the OCTET technique,

or one of its derivative compounds or functional fragments of said antibody,

said antibody in particular being a chimeric antibody or a humanized antibody.

Advantageously, the aforementioned antibody is an antibody against the peptide of amino acid sequence SEQ ID NO: 25, said peptide corresponding to native IL-1RA.

The invention is based on the observation made by the inventors that certain specific monoclonal antibodies are capable, due to their very high affinity, of blocking the competitive action of IL-1RA with respect to IL-1, thus making it possible, at a very low concentration, to shift the balance of the IL-1RA/IL-1R interaction toward an IL-1/IL-1R interaction.

The aforementioned antibody is also able to recognize the protein comprising the leader peptide and consisting of the amino acid sequence SEQ ID NO: 26.

The aforementioned antibody can also recognize the different isoforms of IL-1RA, in particular of sequence SEQ ID NO: 33 (isoform 2: MALETICRPS GRKSSKMQA FRIWDVNQKT FYLRNNQLVAG YLQGPNVNLEEKI DWPIEPHALF LGIHGGKMCLS CVKSGDETRLQL EAVNITDLSENR KQDKRFAFIRSD SGPTTSFESAAC PGWFLCTAMEAD QPVSLTNMPDEGV MVTKFYFQEDE), SEQ ID NO: 34 (isoform 3: MALADLYEEG GGGGGEGEDN ADSKETICRP SGRKSSKMQA FRIWDVNQKT FYLRNNQLVA GYLQGPNVNL EEKIDWPIEP HALFLGIHGGKM CLSCVKSGDET RLQLEAVNITDL SENRKQDKRFA FIRSDSGPTTSF ESAACPGWFLC TAMEADQPVS LTNMPDEGVM VTKFYFQEDE) and SEQ ID NO: 35 (isoform 4: MQAFRIWDVNQ KTFYLRNNQLV AGYLQGPNVNLE EKIDWPIEPHA LFLGIHGGKMC LSCVKSGDETR LQLEAVNITDL SENRKQDKRFA FIRSDSGPTTSF ESAACPGWFLC TAMEADQPVSL TNMPDEGVMVT KFYFQEDE).

IL-1RA has competitive properties with interleukin-1 (IL-1) and in particular with the two IL-1α and IL-1β analogues, so that IL-1 can no longer interact with its membrane-binding receptor, the IL-1R subunit. The antibody according to the invention also has competition properties, such that when the antibody recognizes and interacts with IL-1RA, the antibody IL-1RA complex is not capable of interacting with the receptor. There is therefore an inhibition of the interaction between IL-1RA and IL-1R.

The aforementioned antibody has a very strong affinity for I IL-1RA, this affinity, defined by the constant KD of less than 10⁻⁸ M, in particular of the order of 10⁻⁹ M.

In the invention, “of the order of 10⁻⁹ M” means values of 4·10⁻⁹ M to 0.6·10⁻⁹ M.

The term “KD” refers to the equilibrium dissociation constant of a particular protein-antigen interaction. Typically, the antibody of the invention binds to its target antigen with an equilibrium dissociation constant (KD) of less than about 10⁻⁸ M or less than 10⁻⁹ M or even less than 10⁻¹⁰ M or even lower.

There are several techniques for determining the affinity of one protein with another. Examples include the BIACORE method, which uses surface plasmon resonance (SPR), the OCTET method, interaction monitoring techniques using radiolabeling of a protein radiolabeled with Iodine-125 or its synthesis incorporating a radiolabeled amino acid.

The OCTET method used in the context of the invention is based on the technology of biological layer interferometry (BLI), which allows the real-time analysis, without label, of the affinity as well as of the kinetics. The principle of BLI technology is based on the optical interference pattern of white light reflected from two surfaces—an immobilized protein layer and an internal reference layer. Binding between an immobilized ligand on the surface of the biosensor tip and an analyte in solution produces an increase in optical thickness at the biosensor tip, which results in a change in the interference pattern measured in nanometers. The wavelength shift (AA) is a direct measure of the change in optical thickness of the biolayer, when this shift is measured over a period of time and its magnitude is plotted against time, a curve of classical association/dissociation is obtained. This interaction is measured in real time, providing the ability to monitor binding specificity, association rate and dissociation rate, and concentration with high precision and accuracy.

Within the meaning of the present invention, the term “antibody” designates immunoglobulin molecules, regardless of the class of mouse immunoglobulin (IgD, IgM, IgG1, IgG2a, IgG2b, IgG3, IgE or IgA) or human immunoglobulin (IgD, IgM, IgG1, IgG2, IgG3, IgG4, IgE, IgA1 or IgA2) or any other equivalent or similar classes of other species such as rats, rabbits or llamas, for example, and immunologically active portions of molecules of immunoglobulin, i.e. molecules that contain a binding site that binds specifically to an antigen, whether natural or partially or entirely produced by synthesis. The term also covers any polypeptide or protein or polycarbonate molecule having a binding domain that is, or is homologous to, an antibody binding domain. These can come from natural sources, or else be produced in part or in whole by synthesis.

An antibody is a molecule made up of two heavy polypeptide chains and two light polypeptide chains linked together by disulfide bridges between cysteines. Each heavy chain is composed of a variable domain (“VH domain”) and 3 constant domains (“CH1,” “CH2” and “CH3” domains), while each light chain is composed of a variable domain (“VL domain”) and a constant domain (“CL domain”). In addition, each variable domain (VH or VL domain) is composed of 4 “framework domains” (FR1, FR2, FR3 and FR4) that exhibit less sequence variability among antibodies and are involved in the formation of beta sheets, and 3 hypervariable domains, commonly called “complementarity-determining domains” 1, 2 and 3 (CDR1, CDR2, CDR3) that correspond to 3 loops separating each beta sheet. The 3 CDR domains, juxtaposed in space at the end of the variable domain, are crucial for the determination of an antibody (or antibody fragment) specificity since they are the part of the variable domain mainly in contact with the antigen, in particular the CDR3 domain of each chain, corresponding to the rearranged domain of the heavy and light chains, which is even more variable and more directly in contact with the specific antigen.

Examples of antibodies are immunoglobulin isotypes (e.g., IgG, IgE, IgM, IgD and IgA) and their isotypic subclasses.

Since antibodies can be modified in a number of ways, the term “antibody” should be interpreted to cover any specific binding member or substance having a binding domain with the required specificity. Thus, this term covers fragments of antibodies, derivatives, functional equivalents and homologs of antibodies, humanized or chimeric antibodies, including any polypeptide comprising an immunoglobulin binding domain, as well as any polycarbonate molecule having a similar capacity, whether natural or completely or partly synthetic. Chimeric molecules comprising an immunoglobulin binding domain, or functional equivalent, fused to another polypeptide are therefore covered by the definition. The present invention also covers any mineral, organic or polypeptide molecule capable of modulating the transcription, expression or protein synthesis of IL-1RA produced in the native state by cells or tissues, human in particular.

The monoclonal antibodies of the invention can be humanized by altering the amino acid sequence of the antibody. Humanization comprises grafting CDRs onto a suitable antibody framework or remodeling residues of varying surface area, e.g., by site-directed mutagenesis or other commonly used molecular biology techniques. A humanized antibody can be a modified antibody having the variable domains of a non-human antibody, e.g., murine, and the constant domain of a human antibody.

It has been shown that fragments of a whole antibody can perform the function of binding to the antigen. Examples of antibody fragments are:

i. the Fab fragment consisting of the VL, VH, CL and CH1 domains;

i. the Fd fragment consisting of the VH and CH1 domains;

iii. the Fv fragment consisting of the VL and VH domains of a single antibody or a dsFv fragment;

iv. the dAb fragment, which consists of a VH domain;

v. isolated CDR domains

vi. the F(ab′) fragment obtained from an F(ab′)2 fragment by cleavage of the disulfide bridge in the hinge domain;

vii. F(ab′)2 fragments, a bivalent fragment comprising two linked Fab fragments;

viii. single chain variable fragments (scFv), in which a VH domain and a VL domain are linked by a peptide linker that allows the two domains to combine to form an antigen binding site;

ix. bispecific single-stranded Fv dimers;

x. “diabodies,” multivalent or multispecific fragments constructed by gene fusion;

xi. an scFv molecule.

All of these antibody fragments and derivatives are well known to those skilled in the art.

Advantageously, the invention relates to the aforementioned antibody, recognizing a conformational or linear epitope of IL-1RA, said conformational or linear epitope comprising at least one of the following amino acid sequences: FRIWDVNQKT (SEQ ID NO: 36), GYLQGPNVNL (SEQ ID NO: 37), DSGPTT (SEQ ID NO: 38), AMEADQ (SEQ ID NO: 39), NMPDEGVMVTKFYFQED (SEQ ID NO: 40), FYLRNNQL (SEQ ID NO: 41), LQGPVNLEEK (SEQ ID NO: 42) and HGGGMCL (SEQ ID NO: 43), all these sequences being comprised in the sequence SEQ ID NO: 25.

Advantageously, said conformational or linear epitope comprises at least one of the amino acid sequences belonging to the following amino acid sequence SEQ ID NO: 25: SEQ ID NO: 36, SEQ ID NO: 37, SEQ ID NO: 38, SEQ ID NO: 39, and SEQ ID NO: 40, all these sequences being included in sequence SEQ ID NO: 25.

In particular, said conformational or linear epitope comprises at least the amino acid sequences SEQ ID NO: 36, and SEQ ID NO: 40 belonging to amino acid sequence SEQ ID NO: 25. The binding of an antibody to such an epitope advantageously makes it possible to block the interaction between IL-1RA and the IL-1 receptor. Indeed, these two sequences cover amino acids belonging to the two IL-1RA binding sites by which IL-1RA binds to the IL-1 receptor. Advantageously, said conformational or linear epitope comprises at least the amino acid sequences SEQ ID NO: 36, SEQ ID NO: 39, and SEQ ID NO: 40 belonging to amino acid sequence SEQ ID NO: 25. In particular again, said conformational or linear epitope additionally comprises amino acid sequence SEQ ID NO: 37 belonging to amino acid sequence SEQ ID NO: 25. Such an epitope thus covers all the amino acids of the two binding sites of IL-1RA by which the latter binds to the IL-1 receptor.

In particular, said conformational or linear epitope can be formed by
amino acid sequences SEQ ID NO: 36, SEQ ID NO: 38, SEQ ID NO: 39, and SEQ ID NO: 40 belonging to amino acid sequence SEQ ID NO: 25, or
amino acid sequences SEQ ID NO: 36, SEQ ID NO: 37, SEQ ID NO: 39, and SEQ ID NO: 40 belonging to amino acid sequence SEQ ID NO: 25, or
amino acid sequences SEQ ID NO: 36, SEQ ID NO: 37, SEQ ID NO: 38, SEQ ID NO: 39, and SEQ ID NO: 40 belonging to amino acid sequence SEQ ID NO: 25, or
amino acid sequence SEQ ID NO: 37 belonging to amino acid sequence SEQ ID NO: 25, or
amino acid sequences SEQ ID NO: 41, SEQ ID NO: 42 and SEQ ID NO: 43 belonging to amino acid sequence SEQ ID NO: 25.

In the invention, “epitope” means one or more domains of the amino acid sequence of the target protein recognized by the antibody.

In the invention, “linear epitope” means an epitope whose domain(s) of the target protein are recognized by the antibody whether said protein is in three-dimensional or denatured form. It is therefore the sequence as such that is important for the recognition of the antibody.

In the invention, “conformational epitope” means an epitope exhibiting a determined three-dimensional structure, this three-dimensional structure and the sequences of the protein that are found there allowing recognition by the antibody. Thus, a conformational epitope is formed by amino acids that are not necessarily contiguous in the sequence of the target protein, in this case the sequence of IL-1RA and its isoforms (SEQ ID NO. 25, 26, 33, 34, 35). Unlike a linear epitope, the domain(s) of the target protein are not recognized by the antibody in the event of denaturation of said protein. Here, it is therefore both the sequence as such, and also its structuring in space, that are important for the recognition of the antibody.

Advantageously, the invention relates to the aforementioned antibody, comprising a light chain comprising at least one CDR chosen from CDR-L1, CDR-L2 and CDR-L3, where
CDR-L1 comprises one of the sequences SEQ ID NO: 1, SEQ ID NO: 4 and SEQ ID NO: 7,
CDR-L2 comprises one of the sequences SEQ ID NO: 2, SEQ ID NO: 5, and SEQ ID NO: 8,
CDR-L3 comprises one of the sequences SEQ ID NO: 3, SEQ ID NO: 6 and SEQ ID NO: 9, or one of its derivative compounds or functional fragments.

Within the meaning of the present invention, the term “CDR” means “complementarity-determining regions” (also referred to as “complementarity-determining domains”) and is used to designate an amino acid sequence of a heavy chain or light chain variable domain. CDRs are required for binding to the antigen, and determine the specificity of the antibody. Each variable domain typically comprises three CDRs identified as CDR1 (CDR-H1 or CDR-L1, where “H” indicates the heavy chain CDR1 and “L” indicates the light chain CDR1), CDR2 (CDR-H2 or CDR-L2, where “H” indicates the heavy chain CDR2 and “L” the light chain CDR2) and CDR3 (CDR-H3 or CDR-L3, where “H” indicates the heavy chain CDR3 and “L” the light chain CDR3).

In the invention, “comprising a light chain comprising at least one CDR chosen from CDR-L1, CDR-L2 and CDR-L3” means that the antibody comprises at least one of CDR-L1, CDR-L2 and CDR-L3 as defined above, or two of them or all three.

Of course, all the aforementioned fragments are covered by this advantageous embodiment.

Advantageously, the antibody comprises three of the aforementioned CDRs, i.e. a CDR-L1, a CDR-L2 and a CDR-L3. Referring to the aforementioned sequences, the possible combinations of three CDRs are as follows:
SEQ ID NO: 1, SEQ ID NO: 2, and SEQ ID NO: 3,
SEQ ID NO: 1, SEQ ID NO: 2, and SEQ ID NO: 6,
SEQ ID NO: 1, SEQ ID NO: 2, and SEQ ID NO: 9,
SEQ ID NO: 1, SEQ ID NO: 5 and SEQ ID NO: 3,
SEQ ID NO: 1, SEQ ID NO: 5 and SEQ ID NO: 6,
SEQ ID NO: 1, SEQ ID NO: 5 and SEQ ID NO: 9,
SEQ ID NO: 1, SEQ ID NO: 8 and SEQ ID NO: 3,
SEQ ID NO: 1, SEQ ID NO: 8 and SEQ ID NO: 6,
SEQ ID NO: 1, SEQ ID NO: 8 and SEQ ID NO: 9,
SEQ ID NO: 4, SEQ ID NO: 2, and SEQ ID NO: 3,
SEQ ID NO: 4, SEQ ID NO: 2, and SEQ ID NO: 6,
SEQ ID NO: 4, SEQ ID NO: 2, and SEQ ID NO: 9,
SEQ ID NO: 4, SEQ ID NO: 5 and SEQ ID NO: 3,
SEQ ID NO: 4, SEQ ID NO: 5 and SEQ ID NO: 6,
SEQ ID NO: 4, SEQ ID NO: 5 and SEQ ID NO: 9,
SEQ ID NO: 4, SEQ ID NO: 8 and SEQ ID NO: 3,
SEQ ID NO: 4, SEQ ID NO: 8 and SEQ ID NO: 6,
SEQ ID NO: 4, SEQ ID NO: 8 and SEQ ID NO: 9,
SEQ ID NO: 7, SEQ ID NO: 2, and SEQ ID NO: 3,
SEQ ID NO: 7, SEQ ID NO: 2, and SEQ ID NO: 6,
SEQ ID NO: 7, SEQ ID NO: 2, and SEQ ID NO: 9,
SEQ ID NO: 7, SEQ ID NO: 5 and SEQ ID NO: 3,
SEQ ID NO: 7, SEQ ID NO: 5 and SEQ ID NO: 6,
SEQ ID NO: 7, SEQ ID NO: 5 and SEQ ID NO: 9,
SEQ ID NO: 7, SEQ ID NO: 8 and SEQ ID NO: 3,
SEQ ID NO: 7, SEQ ID NO: 8 and SEQ ID NO: 6, and
SEQ ID NO: 7, SEQ ID NO: 8 and SEQ ID NO: 9.

Advantageously, the invention relates to the aforementioned antibody, comprising a heavy chain comprising at least one CDR chosen from CDR-H1, CDR-H2 and CDR-H3, where
CDR-H1 comprises one of the sequences SEQ ID NO: 10 (EYTMH), SEQ ID NO: 13 and SEQ ID NO: 16,
CDR-H2 comprises one of the sequences SEQ ID NO: 11, SEQ ID NO: 14 and SEQ ID NO: 17,
CDR-H3 comprises one of the sequences SEQ ID NO: 12, SEQ ID NO: 15 and SEQ ID NO: 18, or one of its derivative compounds or functional fragments.

Again all of the aforementioned fragments are covered by this advantageous embodiment.

Advantageously, the antibody comprises three of the aforementioned CDRs, i.e. a CDR-H1, a CDR-H2 and a CDR-H3. Referring to the aforementioned sequences, the possible combinations of three CDRs are as follows:
SEQ ID NO: 10, SEQ ID NO: 11 and SEQ ID NO: 12,
SEQ ID NO: 10, SEQ ID NO: 11 and SEQ ID NO: 15,
SEQ ID NO: 10, SEQ ID NO: 11 and SEQ ID NO: 18,
SEQ ID NO: 10, SEQ ID NO: 14 and SEQ ID NO: 12,
SEQ ID NO: 10, SEQ ID NO: 14 and SEQ ID NO: 15,
SEQ ID NO: 10, SEQ ID NO: 14 and SEQ ID NO: 18,
SEQ ID NO: 10, SEQ ID NO: 17 and SEQ ID NO: 12,
SEQ ID NO: 10, SEQ ID NO: 17 and SEQ ID NO: 15,
SEQ ID NO: 10, SEQ ID NO: 17 and SEQ ID NO: 18,
SEQ ID NO: 13, SEQ ID NO: 11 and SEQ ID NO: 12,
SEQ ID NO: 13, SEQ ID NO: 11 and SEQ ID NO: 15,
SEQ ID NO: 13, SEQ ID NO: 11 and SEQ ID NO: 18,
SEQ ID NO: 13, SEQ ID NO: 14 and SEQ ID NO: 12,
SEQ ID NO: 13, SEQ ID NO: 14 and SEQ ID NO: 15,
SEQ ID NO: 13, SEQ ID NO: 14 and SEQ ID NO: 18,
SEQ ID NO: 13, SEQ ID NO: 17 and SEQ ID NO: 12,
SEQ ID NO: 13, SEQ ID NO: 17 and SEQ ID NO: 15,
SEQ ID NO: 13, SEQ ID NO: 17 and SEQ ID NO: 18,
SEQ ID NO: 16, SEQ ID NO: 11 and SEQ ID NO: 12,
SEQ ID NO: 16, SEQ ID NO: 11 and SEQ ID NO: 15,
SEQ ID NO: 16, SEQ ID NO: 11 and SEQ ID NO: 18,
SEQ ID NO: 16, SEQ ID NO: 14 and SEQ ID NO: 12,
SEQ ID NO: 16, SEQ ID NO: 14 and SEQ ID NO: 15,
SEQ ID NO: 16, SEQ ID NO: 14 and SEQ ID NO: 18,
SEQ ID NO: 16, SEQ ID NO: 17 and SEQ ID NO: 12,
SEQ ID NO: 16, SEQ ID NO: 17 and SEQ ID NO: 15, and
SEQ ID NO: 16, SEQ ID NO: 17 and SEQ ID NO: 18.

Advantageously, the invention relates to the aforementioned antibody, or fragments thereof, said antibody comprising a light chain comprising:
the CDRs of sequences SEQ ID NO: 1 to 3, or
the CDRs of sequences SEQ ID NO: 4 to 6, or
the CDRs of sequences SEQ ID NO: 7 to 9.

Advantageously, the invention relates to the aforementioned antibody, or fragments thereof, said antibody comprising a heavy chain comprising:
the CDRs of sequences SEQ ID NO: 10 to 12, or
the CDRs of sequences SEQ ID NO: 13 to 15, or
the CDRs of sequences SEQ ID NO: 16 to 18.

Even more advantageously, the invention relates to the aforementioned antibody, said antibody comprising.
a variable part of the light chain comprising any one of the following sequences SEQ ID NO: 19 to 21, and
a variable part of the heavy chain comprising any one of the following sequences

SEQ ID NO: 22 to 24.

The advantageous combination of a light chain and a heavy chain, which confers the aforementioned properties to said antibody, can be one of the following combinations:
a variable part of the light chain of sequence SEQ ID NO: 19 and a variable part of the heavy chain of sequence SEQ ID NO: 22,
a variable part of the light chain of sequence SEQ ID NO: 19 and a variable part of the heavy chain of sequence SEQ ID NO: 23,
a variable part of the light chain of sequence SEQ ID NO: 19 and a variable part of the heavy chain of sequence SEQ ID NO: 24,
a variable part of the light chain of sequence SEQ ID NO: 20 and a variable part of the heavy chain of sequence SEQ ID NO: 22,
a variable part of the light chain of sequence SEQ ID NO: 20 and a variable part of the heavy chain of sequence SEQ ID NO: 23,
a variable part of the light chain of sequence SEQ ID NO: 20 and a variable part of the heavy chain of sequence SEQ ID NO: 24,
a variable part of the light chain of sequence SEQ ID NO: 21 and a variable part of the heavy chain of sequence SEQ ID NO: 22,
a variable part of the light chain of sequence SEQ ID NO: 21 and a variable part of the heavy chain of sequence SEQ ID NO: 23, and
a variable part of the light chain of sequence SEQ ID NO: 21 and a variable part of the heavy chain of sequence SEQ ID NO: 24.

Advantageously, said antibody
comprises a variable part of the light chain of sequence SEQ ID NO: 19 and a variable part of the heavy chain of sequence SEQ ID NO: 22, or
comprises a variable part of the light chain of sequence SEQ ID NO: 20 and a variable part of the heavy chain of sequence SEQ ID NO: 23, or
comprises a variable part of the light chain of sequence SEQ ID NO: 21 and a variable part of the heavy chain of sequence SEQ ID NO: 24.

The invention further relates to a nucleic acid molecule encoding a light chain of the monoclonal antibody as defined above.

It is possible to produce the antibody according to the invention by expressing nucleic acid molecules in these appropriate mammalian cells, insect cells or plant cells. Mammalian cells are particularly suitable insofar as they allow glycosylation of the constant parts of the antibodies that they synthesize.

In a particularly advantageous manner, the antibody of the invention comprises a light chain comprising a variable sequence encoded by one of the nucleic acid sequences SEQ ID NO: 27, SEQ ID NO: 29 or SEQ ID NO: 31.

The invention further relates to a nucleic acid molecule encoding a heavy chain of the monoclonal antibody as defined above.

In a particularly advantageous manner, the antibody of the invention comprises a heavy chain comprising a variable sequence encoded by one of the nucleic acid sequences SEQ ID NO: 28, SEQ ID NO: 30 or SEQ ID NO: 32.

Advantageously, the preferred antibodies of the invention are encoded by
a first nucleic acid sequence comprising a sequence encoding the variable part of the light chain SEQ ID NO: 27 and a second nucleic acid sequence comprising a sequence encoding the variable part of the heavy chain SEQ ID NO: 27,
a first nucleic acid sequence comprising a sequence encoding the variable part of the light chain SEQ ID NO: 29 and a second nucleic acid sequence comprising a sequence encoding the variable part of the heavy chain SEQ ID NO: 30,
a first nucleic acid sequence comprising a sequence encoding the variable part of the light chain SEQ ID NO: 31 and a second nucleic acid sequence comprising a sequence encoding the variable part of the heavy chain SEQ ID NO: 32.

In another aspect, the invention relates to a pharmaceutical composition comprising an antibody as defined above, in association with a pharmaceutically acceptable vehicle, or comprising a nucleic acid molecule as defined above, in association with a pharmaceutically acceptable vehicle.

The invention further relates to a pharmaceutical composition comprising the antibody according to the invention as active principle and at least one pharmaceutically acceptable carrier. In a particular embodiment of the invention, the pharmaceutical composition according to the invention comprises a therapeutically effective amount of the antibody as active principle and at least one pharmaceutically acceptable carrier.

Within the meaning of the present invention, the terms “therapeutically effective” or “amount effective to treat” or “pharmaceutically effective” designate the amount of antibody necessary to inhibit or reverse a disease. The determination of a therapeutically effective amount depends specifically on factors such as the toxicity and effectiveness of the drug. These factors will differ depending on other factors such as activity, relative bioavailability, patient body weight, severity of unwanted side effects, and preferred method of administration. Toxicity can be determined using methods well known in the art. The same is true for effectiveness. A pharmaceutically effective amount is, therefore, an amount that is considered by the clinician to be toxicologically tolerable, but effective.

The dosage can be adjusted as appropriate to achieve desired levels of drug (anti-IL-1RA antibody or antibody fragment), local or systemic, depending on the mode of administration. In the event that the response in a patient is insufficient at such doses, even higher doses (or doses more effective by a different, more localized route of administration) may be used as long as the tolerance of the patient allows it. Multiple doses per day can also be used to achieve appropriate levels of antibody or antibody fragments. “Dose” and “dosage” are used interchangeably herein.

In an advantageous embodiment, the amount of the antibody contained in the pharmaceutical composition administered to the patient is from 2 mg/kg to approximately 20 mg/kg of body weight, or from approximately 110 mg to approximately 1.6 g by injection for a person of from 55 to 80 kg in body weight. In an even more advantageous embodiment, the dosage of the human IL-1RA antibody or the antibody fragment is in the range of approximately 0.5 mg/kg to approximately 5 mg/kg of body weight, or approximately 27.5 mg to approximately 0.4 g per injection. In an even more advantageous embodiment, the dosage of the antibody is in the range of approximately 0.2 mg/kg to approximately 2 mg/kg of body weight, or approximately 11 mg to approximately 0.16 g per injection. The frequency of injection is linked to the half-life of the antibody.

Preferably, the pharmaceutical compositions are administered in unit dosage forms suitable for single administration of precise dosage amounts. The pharmaceutical compositions can also comprise, depending on the desired formulation, at least one pharmaceutically acceptable carrier or diluent, which are defined as aqueous-based vehicles commonly used for formulations intended for animal or human administration. The diluent is selected so as not to affect the biological activity of the active compounds present in the pharmaceutical combination of the invention. Examples of such diluents are distilled water, physiological saline, Ringer's solution, dextrose solution and Hanks' solution. The same diluents can be used to reconstitute the lyophilized pharmaceutical composition of interest. Furthermore, the pharmaceutical composition may also comprise other medicinal agents (antibiotics, antivirals, antiparasitics, antifungals, for example), pharmaceutical agents, carriers, adjuvants or non-toxic, non-therapeutic or non-immunogenic stabilizers, etc. Effective amounts of such diluent or carrier are amounts effective to obtain a pharmaceutically acceptable formulation in terms of solubility of the components and biological activity, etc. In some embodiments, the pharmaceutical compositions provided herein are sterile. Administration of the pharmaceutical composition can be by any route of administration, including oral, topical, subcutaneous, parenteral, intramuscular, intranasal, sublingual, intratracheal, inhalation, ocular, vaginal and rectal. Intracapsular, intravenous and intraperitoneal routes of administration can also be used. A person skilled in the art will know how to select the appropriate route of administration depending on the problem to be treated.

For example, the composition or specific binding protein according to the present invention can be administered to a subject by oral, subcutaneous, parenteral or topical administration. In one embodiment, the compositions or the antibody of the present invention are administered by intravenous infusion. The compositions, when it is desirable to be administered systemically, can be formulated for parenteral administration by injection, for example, by bolus injection or continuous infusion. Formulations for injection may be presented in unit dosage form, for example in ampoules or in multiple dose containers, with a preservative added. The compositions can take forms such as suspensions, solutions or emulsions in oily or aqueous vehicles, and can contain formulating agents such as suspending agents, stabilizers and/or dispersants.

The invention further relates to the aforementioned antibody as a drug.

The invention also relates to the aforementioned pharmaceutical composition as a drug.

The invention further relates to a composition comprising
an antibody as defined above, or
the nucleic acid molecules as defined above,
or a mixture thereof,
for the treatment of inflammatory, infectious or autoimmune diseases, in particular for the treatment of Lyme disease, in particular chronic forms of Lyme disease.

Within the meaning of the present invention, the term “treatment” or “curative treatment” is defined as a treatment leading to a cure or a treatment that alleviates, improves and/or eliminates, reduces and/or stabilizes the symptoms of a disease or the suffering it causes.

The inflammatory, infectious or autoimmune diseases are advantageously diseases linked to an expression or an induction of the abnormal expression of IL-1RA. This means that the inhibition of cell signaling in response to IL-1 caused by an excessive presence of IL-1RA will be totally or partially inhibited by the presence of the antibody according to the invention. Examples of inflammatory, infectious or autoimmune diseases include alcoholic hepatic fibrosis, bone loss during early menopause, rheumatoid arthritis, type I and II diabetes, Crohn's disease, ulcerative colitis, nephropathy, multiple sclerosis, myasthenia gravis, Basdow Grave's autoimmune thyroiditis, microvascular coronary disease, lupus, Gougerot-Sjögren's syndrome, alopecia areata, lichen sclerosus, rheumatoid purpura, long and severe forms of malaria, joint pathologies, rheumatoid arthritis, juvenile idiopathic arthritis, chronic fatigue such as fibromyalgia, cancer of the cervix linked to papillomavirus, stomach ulcers caused by Helicobacter Pilori, plague, mycobacteriosis, leprosy, chronic extra-pulmonary and mildly inflammatory forms of tuberculosis, long and delayed forms of COVID-19.

More precisely concerning the expression or the induction of the abnormal expression of IL-1RA, it is an expression or an induction of the abnormal expression of IL-1RA allele 2. At the intron 2 of the gene coding for IL-1RA, there are 5 different alleles (VNTR zone) where a segment of 86 base pairs is repeated from 2 to 6 times (each segment of 86 bp possessing 3 binding sites for transcription elements for the protein). The allele comprises 4 repetitions; it is called “long allele” (IL-1RN*L). It is the most common allele in healthy people (72%). Allele 2 comprises 2 repetitions; it is called “long allele (IL-1RN*2). It is less frequent in healthy people (24%), but corresponds to the most effective allele for protein transcription. It is known to be associated with more or less chronic/acute forms of infectious diseases. Allele 3 has 5 repetitions, allele 4 has 3 repetitions, allele 5 has 6 repetitions. These last three alleles are very infrequent and are present in 4% of healthy people.

An inflammatory disease is caused by the inflammation of an organ. It can affect most organs of the human body and can be caused by a systemic disease, an allergy, an infection or even cancer. Symptom treatments are based on so-called anti-inflammatory drugs, i.e. drugs that limit inflammation.

Autoimmune diseases result from a dysfunction of the immune system leading it to attack normal constituents of the body.

In another aspect, disclosed is a method of treating inflammatory, infectious or autoimmune diseases comprising the step of administering a pharmaceutically active amount to an individual or animal in need of a composition comprising:
an antibody as defined above, or
the nucleic acid molecules as defined above,
or a mixture thereof.

In another aspect, disclosed is a method of treating Lyme disease comprising the step of administering a pharmaceutically active amount to an individual or animal in need of a composition comprising:
an antibody as defined above, or
the nucleic acid molecules as defined above, or
a mixture thereof.

In another aspect, disclosed is a method of treating the chronic or complex form of Lyme disease comprising the step of administering a pharmaceutically active amount to an individual or animal in need of a composition comprising:
an antibody as defined above, or
the nucleic acid molecules as defined above, or
a mixture thereof.

The invention further relates to an antibody as defined above, for its use for the diagnosis of inflammatory, infectious or autoimmune diseases, in particular the diagnosis of Lyme disease, in particular the diagnosis of chronic forms of Lyme disease.

The antibody according to the invention insofar as it interacts with IL-1RA is capable of detecting the amount, the absence or the presence of IL-1RA and of making it possible to make a diagnosis of a pathology involving dysregulation of this antagonist.

The antibody can be used in this diagnostic context alone, or in a form conjugated with a molecule allowing luminescent, luminous or phosphorescent detection. The antibody can also be used with another antibody recognizing IL-1RA, without this second antibody necessarily having blocking properties like the antibody of the invention.

The antibody according to the invention can also be used in the context of a diagnosis in combination with one or more other antibodies recognizing the constant parts of the antibodies (including the antibody of the invention).

The invention also relates to a method for the diagnosis, in particular in vivo, of an inflammatory, infectious or autoimmune pathology or disease, comprising:
a step of bringing a biological sample from an individual suspected of suffering from inflammatory, infectious or autoimmune diseases into contact with an antibody as defined above, and
a step for detecting the presence or absence of, or even quantifying, the IL-1RA protein in said sample, and
a step for comparison with at least one reference sample, whether it is a positive control or a negative control or both.

From this comparison, in particular where the quantity of IL-1RA is measured, it is possible to determine whether or not the patient from whom the biological sample is taken is affected by said pathology or disease.

The invention also relates to a method for the diagnosis, in particular in vivo, of Lyme disease, comprising:
a step of bringing a biological sample from an individual suspected of having Lyme disease into contact with an antibody as defined above, and
a step for detecting the presence or absence of, or even quantifying, the IL-1RA protein in said sample, and
a step for comparison with at least one reference sample, whether it is a positive control or a negative control or both.

The invention also relates to a method for the diagnosis, in particular in vivo, of the chronic form of Lyme disease, comprising:
a step of bringing a biological sample from an individual suspected of having the chronic form of Lyme disease into contact with an antibody as defined above, and
a step for detecting the presence or absence of, or even quantifying, the IL-1RA protein in said sample, and
a step for comparison with at least one reference sample, whether it is a positive control or a negative control or both.

The present invention further relates to a diagnostic kit for detecting IL-1RA, preferably in patients suffering or likely to suffer from a disease associated with an abnormal level of IL-1RA, said kit comprising an antibody as defined above, in association with a means for detecting the interaction between said antibody and IL-1RA.

In a particularly advantageous embodiment, the present invention also relates to a diagnostic kit for detecting IL-1RA, preferably in patients suffering or likely to suffer from an inflammatory, autoimmune or infectious disease, said kit comprising an antibody as defined above, in association with a means for detecting the interaction between said antibody and IL-1RA.

In a particularly advantageous embodiment, the present invention also relates to a diagnostic kit for detecting IL-1RA, preferably in patients suffering from or susceptible to Lyme disease, or its chronic form, said kit comprising an antibody as defined above, in association with a means for detecting the interaction between said antibody and IL-1RA.

In a particular embodiment according to the invention, the diagnostic kit comprises the following elements:
the antibody as previously defined, optionally labeled or optionally coupled to a solid support, such as a tissue culture plate or beads, advantageously Sepharose beads;
where appropriate, a reagent for the constitution of the medium conducive for carrying out the immunological reaction;
where appropriate, a reagent making possible the detection of specific antigen-binding protein complexes produced by the immunological reaction, this reagent possibly also carrying a label, or being capable of being recognized in turn by a labeled reagent, more particularly in the case where said antibody is not labeled;
where appropriate, reagents for carrying out lysis of the cells of the sample tested.

Advantageously, chromophoric, fluorescent, radioactive or chemofluorescent reagents may be mentioned as examples of a reagent making possible the detection of specific antigen-antibodies produced by the immunological reaction.

Advantageously, the labeling of the antibody can in particular be carried out using a detectable molecule, such as a fluorescent molecule, a radioactive molecule, a radical probe, also called “spin label” for detection by nuclear magnetic resonance (NMR) imaging or any other type of molecule well known to a person skilled in the art. By way of example of a radioactive molecule, mention may in particular be made of iodine 123, iodine 125, indium 111, rhenium 186, fluorine 19, carbon 13, nitrogen 15, oxygen 17, gadolinium, manganese or iron, the list not being exhaustive.

Advantageously, the antibody can also be coupled to a molecule making it possible to prolong its half-life in the body, such as polyethylene glycol

Advantageously, a person skilled in the art will know how to select the reagents necessary to carry out lysis of the cells of the biological sample tested.

The invention will be better understood in the light of the following examples and drawings.

### BRIEF DESCRIPTION OF THE FIGURES

FIG. 1 represents a histogram showing the measurement of the interaction between the biotinylated IL-1RA antigen and different doses of commercial anti-IL-1RA control antibody (T+), dilution in cascades of 5 mouse sera (S1 to S5) of a negative control (serum relating to another antibody; Tac) and bovine albumin.

The Y axis represents the optical density measured at 450 nm. For each sample, the first two columns represent testing with 20 and 10 ng/mL of biotinylated IL-1RA antigen, respectively, and the third column represents testing with the antigen interacting with the other antibody.

FIG. 2 represents a graph showing the number of D10S cells per well as a function of the concentration of the antibody tested. A, B and C respectively represent the best clones selected, namely 10E12, 9E11 and 9G5, respectively.

FIG. 3 shows IL-1-induced IL-6 secretion in in vitro cultures of human MG63 osteosarcoma. This synthesis, totally inhibited by IL-1RA, can be restored by adding increasing concentrations of monoclonal antibodies directed against IL-1RA. This restoration of IL-6 secretion then reflects the ability of monoclonals to neutralize IL-1RA.

FIG. 4 represents the measurement curves of the interaction between IL-1RA and the antibodies according to the invention by the OCTET method. As a control, an antibody that does not recognize IL-1RA is used.

FIG. 5 represents graphs showing the absence of cross-reactivity of several pairs of antibodies of the invention with IL-1α, IL-1β, the soluble IL-1RI and RII receptors and the soluble IL-1RAcP co-receptor. From top to bottom, the curves correspond to the fixed 908.9G5/biotinylated 908.9E11; fixed 908.10G3/biotinylated 908.9E11; fixed 908.9G5/biotinylated 908.10E12; fixed 908.10G3/biotinylated 908.10E12 and fixed 908.10G3/biotinylated 908.11H10 pairs.

FIG. 6 represents graphs showing the detection by antibody pairs of recombinant IL-1RA or its native form produced by human THP1 cells stimulated with phorbol 12-myristate 13-acetate (PMA) (isolated bars in light gray). From top to bottom, the biotinylated antibody is biotinylated 903.8D8, biotinylated 908.9E11, biotinylated 908.9G5, and biotinylated 908.10E12.

FIG. 7 represents, in 3 dimensions, the binding sites (A and B) of IL-1RA (3) with the IL-1 receptor (1).

FIG. 8 shows epitope maps of antibodies 908.9E11B6, 908.10E12C1, 908.9G5B9, 903.8D8B1 and 908.11A2H9. For each epitope map, the amino acid sequence is represented of IL-1RA (SEQ ID NO: 48) on which the amino acids are highlighted according to their probability of being part of the epitope recognized by the given antibody. The numbers 1, 61 and 126 represent the position of the first amino acid in each row relative to the amino acid sequence of IL-1RA (SEQ ID NO: 48). No highlighting indicates that the amino acid is not part of the epitope. A light highlight indicates that there is a low probability that the amino acid is part of the epitope. A dark highlight indicates that there is a very high probability that the amino acid is part of the epitope recognized by the given antibody. The amino acids topped by a star correspond to the amino acids belonging to the binding sites of IL-1RA with the IL-1 receptor. The boxes represent common sequences (denoted from I to V) to the epitopes of the various antibodies presented. If the box is in a solid line, the sequence is part of the epitope for the antibody considered. If the box is dotted, the sequence is not part of the epitope. To facilitate reading of the figure, the numbering of the common sequences only appears in relation to the 908.9E11B6 antibody, but it applies to all the boxes represented.

FIG. 9 shows sequence alignments of IL-1RA and its 3 isoforms. The boxes correspond to the common sequences I to V of the epitopes of the different antibodies.

FIG. 10 is a graph representing the ratio (unitless) of IL-1RA/interferon-γ concentrations (in pg/mL) as a function of different groups of individuals (A to D). The individuals of group A are healthy individuals corresponding to the control. Group B individuals are patients who have just been bitten by a tick infected with Borrelia, with photo of erythema migrans, which is a pathognomonic sign of the acute forms of Lyme disease. Individuals in group C are patients with an acute form of Lyme disease who relapsed from the initial treatment). Group D individuals are patients developing remote Lyme disease in a chronic form. On the graph: n represents the number of individuals in each group; M represents the value of the mean of the ratio of IL-1RA/interferon-γ concentrations for each group; p represents the statistical significance obtained when statistically comparing the M value of two groups.

### DETAILED DESCRIPTION

### Examples

### Example 1: Production and Characterization of Murine Anti-IL-1RA Antibody

Immunization

BALB/C mice were immunized by injection into the footpads five times (weekly injections) with recombinant IL-1RA of sequence SEQ ID NO: 25.

Serum Test

The screening is carried out using the ELISA technique with a biotinylated IL-1RA antigen. The IL-1RA antigen is used at 20 ng and 10 ng/well.

The sera are diluted from 1/200 to 1/1600 with a dilution factor of 2, at the rate of 100 μL/well.

The incubation time is 1 hour at room temperature.

Serum from a mouse immunized with an antigen unrelated to IL-1RA is used as a negative control.

The anti-IL-1 RA antibody MAB280 from R&D Systems is used as a positive control.

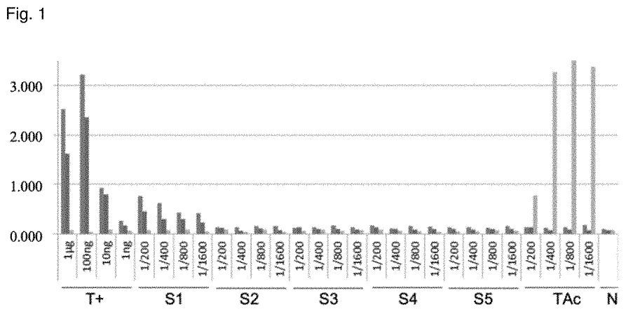

The results obtained are shown in FIG. 1.

Of the 5 sera tested, only serum 1 gives a signal. This signal is not very significant (OD less than 1 for a 1/200 dilution with 20 ng/well of biotinylated IL-1RA), but is specific to biotinylated IL-1RA because no signal is observed with a biotinylated antigen unrelated to IL-1RA as a negative control (TAc).

Cell Fusion

At the end of the immunization process, the blood and lymph node cells were fused with the X63/AG.8653 myeloma following conventional cell fusion protocols to obtain hybridomas.

All the fused cells are distributed in 12 96-well plates.

Selection of Clones

Following immunizations and fusions, 16 candidates were selected and cloned.
10 secrete IgG1
1 secretes IgG2a
3 secrete a mixture of IgG1 and Ig2b
1 secretes a mixture of IgG2a and IgM
1 secretes IgG1+ as well as traces of IgG2a and Ig2b.

Only 8 of the 10 IgG1-secreting hybridomas are saved, and clonally isolated, then amplified and frozen for later experiments.

### Example 2: Study of the Biological Activity of Antibodies on Murine T Line D10S

The biological activity of the 8 anti-IL-1RA antibodies was studied on murine T lymphocyte line D10S (ATCC® TIB-224), known to proliferate in the presence of IL-1. The commercial antibody MAB280 from R&D Systems, an antibody supplied as an IL-1RA blocker, was used as a positive control. D1 OS cells, weaned in IL-113 the day before the test, were cultured in the presence of IL-113 (25 pg/mL), IL-1RA (50 ng/mL) and purified anti-IL-1RA antibodies (10, 5, 2.5 and 1.25 μg/mL) for 72h. Cell proliferation was analyzed using the Guava EasyCyte Plus cytometer.

The 8 clones tested are as follows
903.8D8B1
908.9E11B6
908.9G5B9
908.10E12C1
908.10G3E12
908.11A2H9
908.11G7D4
908.11H10C3

The antibodies serving as controls used are as follows:
MAB280: commercial anti-IL-1RA (R&D Systems)
B-D38 (CTRL IgG1)
B-F33 (CTRL IgG2b).

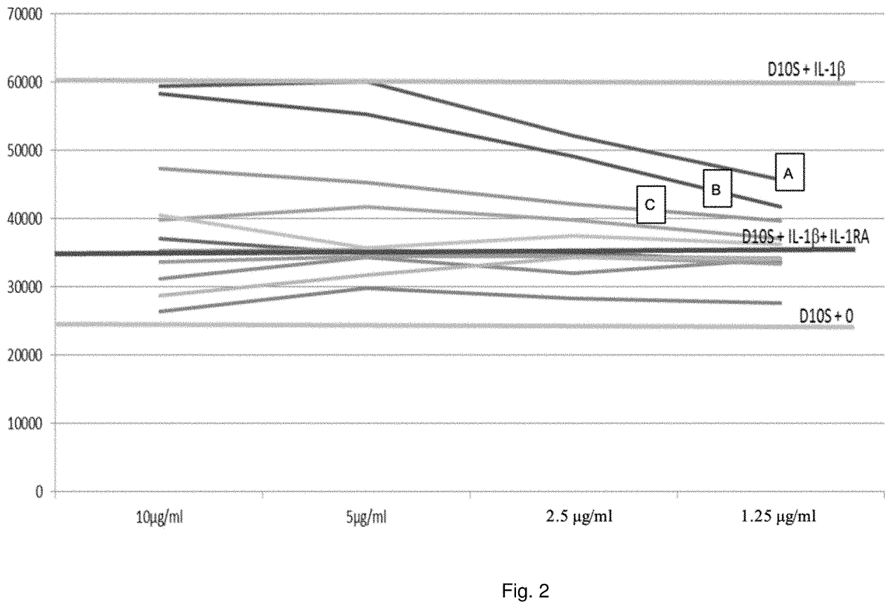

The results obtained are shown in FIG. 2.

The presence of IL-113 increases the proliferation of the D10S line by 2.5 times (line at 60,000). In the presence of IL-1RA, the proliferation of D10S is reduced by a factor of 1.7 (line at 35,000). Proliferation is totally restored in the presence of the 908.10E12C1 (A) and 908.9E11B6 (B) antibodies or partially restored in the presence of the 908.9G5B9 (C) antibody. The other 5 antibodies have little or no blocking activity. The R&D Systems antibody MAB280, described as blocking by the supplier, shows very weak blocking activity in this test. The isotype controls, B-D38 and B-F33, show no neutralizing effect.

Three anti-IL-1RA antibodies exhibit blocking activity of a biological function resulting from the interaction between IL-1β and its receptor:
908.10E12C1 (IgG1)
908.9E11B6 (IgG1),
908.9G5B9 (IgG1).

### Example 3: Study of the Biological Activity of Antibodies on Human Osteosarcoma Cell Line MG63

Human osteosarcoma line MG-63 (ATCC CRL-1427) is known in particular to produce IL-6, easily measured by ELISA technique, in response to treatment with IL-1 (beta or alpha). We therefore selected this line to develop a biological test in which MG-63 is incubated in the presence of IL-1 beta to obtain synthesis of IL-6. Synthesis of IL-6, which will be neutralized by the addition of an excess of IL-1RA, which will compete with IL-1 beta, preventing the latter from acting.

The monoclonal antibodies are then added at different concentrations to measure their ability to neutralize the action of IL-1RA and therefore then allow IL-1 beta to act and induce the synthesis of IL-6. IL-6 which then measured by ELISA. A strong induction of IL-6 will then reflect the blocking nature of the anti-IL-1RA antibody studied.

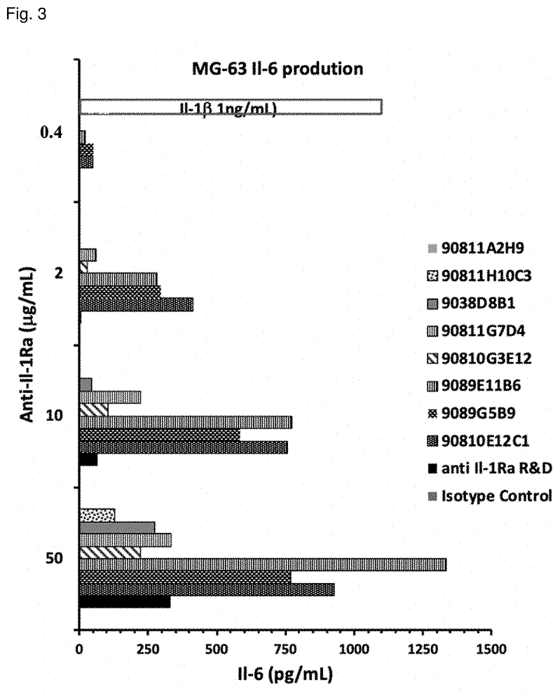

The MG63 cell is seeded in triplicates in 96-well plates with flat bottoms, at the rate of 10,000 per well, under a volume of 100 microliters of RPMI medium+10% fetal calf serum. This is done in the presence of fixed quantities of IL-1 beta (1 ng/mL, R&D Systems) and IL-1RA (100 ng/mL, R&D Systems) and decreasing dilutions of the various anti-IL-RA antibodies ranging from 50 micrograms/mL, 10 micrograms/mL, 2 micrograms/mL to 0.4 micrograms/mL, and appropriate controls, as shown in FIG. 3.

After 48 hours of culture in an incubator at 37° C. and 10% CO2, the culture supernatants are sampled and their IL-6 content assayed by ELISA (R&D Systems), following the recommendations of the kit supplier. The levels of IL-6 measured are expressed in pg/mL as represented on the Y axis of FIG. 3.

All of the 8 antibodies studied show a more or less significant neutralizing activity with respect to IL-1RA. The most potent neutralizing antibodies to IL-1RA are: 9089E 11B6, 9089E G5B9 and 9089E E12C1.

### Example 4: Affinity Study by OCTET

The affinity for the IL-1RA antigen of the 3 blocking antibodies is evaluated using Octet technology on streptavidin biosensors.

The analysis was performed with a 1:1 fit model.

The antibodies used are
908.9E11B6
908.9G5B9
908.10E12C1,
B-D38 (CTRL isotypic IgG1), and
MAB280 (R&D Systems), positive control

The 5 antibodies mentioned above were biotinylated at a 1:1 ratio (1 mole of biotin for 1 mole of antibody) and immobilized on the streptavidin biosensors at a rate of 5 μg/mL.

The IL-1RA antigen has a molecular weight of 17.25 kDa. This antigen was tested at 12 nM at the first point, then diluted 1.5 times on 6 points for the first 4 antibodies. For the MAB280 antibody, this antigen was tested at 50 nM at the first point, then diluted 1.5 times on 5 points.

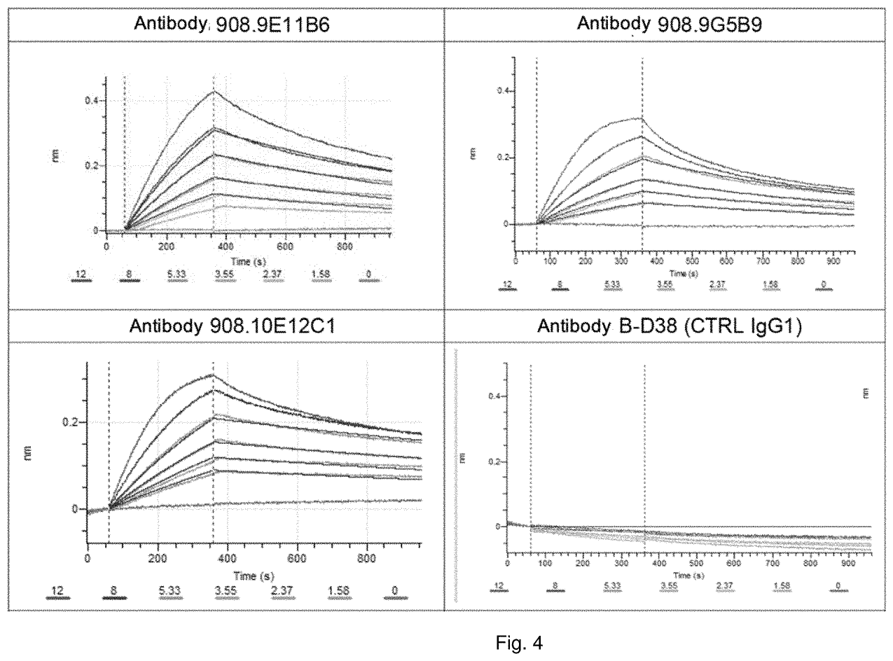

The results are shown in FIG. 4 for the first 4 antibodies and in Table 1 for all antibodies.

For each antibody, 4 concentrations were used to calculate the KD. The constant KD is related to the rate of complex formation (described by the rate constant of association, kon) and the rate of dissociation (described by the rate constant of dissociation, kdis), with KD=kdis/kon. A high affinity interaction is characterized by a low KD, rapid recognition (high kon) and stability of the complexes formed (low kdis). The R2 makes it possible to estimate the reliability of the results obtained (the closer it is to 1, the more reliable the results).

TABLE 1

| | Conc. IL-IRA | | KD | kon | kdis | |
|---|---|---|---|---|---|---|
| Antibody | (nM) | Response | (M) | (1/ms) | (1/s) | R2 |
| 908.9E11B6 | 8 | 0.3128 | 4.86E−09 | 1.78E+05 | 8.65E−04 | 0.9933 |
| 908.9E11B6 | 5.33 | 0.2289 | 4.86E−09 | 1.78E+05 | 8.65E−04 | 0.9933 |
| 908.9E11B6 | 3.55 | 0.1538 | 4.86E−09 | 1.78E+05 | 8.65E−04 | 0.9933 |
| 908.9E11B6 | 2.37 | 0.101 | 4.86E−09 | 1.78E+05 | 8.65E−04 | 0.9933 |
| 908.9G5B9 | 5.33 | 0.2 | 3.84E−09 | 3.42E+05 | 1.31E−03 | 0.9908 |
| 908.9G5B9 | 3.55 | 0.1318 | 3.84E−09 | 3.42E+05 | 1.31E−03 | 0.9908 |
| 908.9G5B9 | 2.37 | 0.092 | 3.84E−09 | 3.42E+05 | 1.31E−03 | 0.9908 |
| 908.9G5B9 | 1.58 | 0.0602 | 3.84E−09 | 3.42E+05 | 1.31E−03 | 0.9908 |
| 908.10E12C1 | 5.33 | 0.21 | 1.76E−09 | 2.64E+05 | 4.64E−04 | 0.9917 |
| 908.10E12C1 | 3.55 | 0.1499 | 1.76E−09 | 2.64E+05 | 4.64E−04 | 0.9917 |
| 908.10E12C1 | 2.37 | 0.107 | 1.76E−09 | 2.64E+05 | 4.64E−04 | 0.9917 |
| 908.10E12C1 | 1.58 | 0.0793 | 1.76E−09 | 2.64E+05 | 4.64E−04 | 0.9917 |
| MAB280 | 50 | 0.1508 | 5.08E−08 | 1.42E+05 | 7.18E−03 | 0.9966 |
| MAB280 | 33.3 | 0.1212 | 5.08E−08 | 1.42E+05 | 7.18E−03 | 0.9966 |
| MAB280 | 22.2 | 0.1047 | 5.08E−08 | 1.42E+05 | 7.18E−03 | 0.9966 |
| MAB280 | 14.8 | 0.0761 | 5.08E−08 | 1.42E+05 | 7.18E−03 | 0.9966 |
| MAB280 | 9.87 | 0.0638 | 5.08E−08 | 1.42E+05 | 7.18E−03 | 0.9966 |

The three anti-IL-1RA antibodies tested all have a KD of the order of 10⁻⁹ M, which means that their affinity for IL-1RA is strong; the 908.10E12C1 antibody appears to be the most affined of the three because its KD is the lowest. The MAB280 antibody has an affinity 10 times lower (greater than 10⁻⁸ M), or even more, than the affinity of the antibodies selected in examples 2, 3 and is therefore not suitable for further development, according to the criteria retained by the pharmaceutical industry.

### Example 5: Cloning

In order to clone the sequences encoding the three antibodies selected in Examples 2, 3 and 4, as well as the 903.8D8B1 and 908.11A2H9 antibodies, the cells that secrete said antibodies were cultured in order to collect the total RNAs.

The RNAs are subjected to two reverse transcription reactions using 5′CDS primers.

The reverse transcription products are then amplified by PCR-RACE using either a primer for the heavy chain or for the light chain.

The PCR products are then cloned into shuttle vectors, for amplification.

The insertion sequences are then sequenced.

Sequencing data was analyzed using the IgBlast database (http://www.ncbi.nlm.nih.gov/igblast/).

The sequences are given below.

The isotype of the antibody from clone 908.9G5 is IgG1/Kappa, that from clone 908.9E11 is IgG1/Lambda, that from clone 10E12 is IgG1/Kappa, that from clone 908.11A2H9 is IgG1/Kappa and that from clone 903.8D8B1 is IgG1/Kappa.

The sequences obtained are described in Table 2 below (the “leader” peptide is in bold):

TABLE 2

| Clone | Chain | SEQ-ID | Sequence |
|---|---|---|---|
| 908.10E12 | Light (VL) | 27 | ATGGAATCACAGACCCAGGTCCTCATGTTTCTTCTGCTCTGGGTAT |
| | | | CTGGTGCCTGTGCAGACATTGTGATGACACAGTCTCCATCCTCCCT |
| | | | GGCTATGTCAGTAGGACAGAAGGTCACTATGAGCTGCAAGTCCAGT |
| | | | CAGAGCCTTTTAAATAGTAGTAATCAAAAGAACTATTTGGCCTGGT |
| | | | ACCAGCAGAAACCAAGACAGTCTCCTAAACTTCTGGTATACTTTGC |
| | | | ATCCACTAGGGATTCTGGGGTCCCTGATCGCTTCTTAGGCAGTGGA |
| | | | TCTGGGACAGATTTCACTCTTACCATCAACCGTGTGCAGGCTGAAG |
| | | | ACCTGGCAGATTACTTCTGTCAGCAACATTATATTCTTCCTCCCAC |
| | | | GTTCGGTGCTGGGACCAAGCTGGAGCTGAAA |
| | Heavy (VH) | 28 | ATGGGATGGAGCTGGATCTTTCTCTTTCTCCTGTCAGGAACTGCAG |
| | | | GTGTCCTCTCTGAGGTCCAGCTGCAACAGTCTGGACCTGAGCTGGT |
| | | | GAAGCCTGGGGCTTCAGTGAAGATATCCTGCAAGACTTCTGGATAC |
| | | | ACATTCACTGAATACACCATGCACTGGGTGAAGCAGAGCCATGGAA |
| | | | AGAGCCTTGAGTGGATTGGAAGTATTAATCCTAACAATGGTGGTAC |
| | | | TAACTACAACCAGAAGTTCAAGGGCAAGGCCACATTGACTGTAGAC |
| | | | AAGTCCTCCAACACAGCCTACATGGAGCTCCGCAGCCTGACATCTG |
| | | | GTGATTCTGCAGTCTATTACTGTGCAAGAACTCTCTACTATGCTAT |
| | | | GGACTACTGGGGTCAAGGAACCTCAGTCACCGTCTCCTCA |
| 908.9E11 | Light (VL) | 29 | ATGGCCTGGATTTCACTTATACTCTCTCTCCTGGCTCTCAGCTCAG |
| | | | GGGCCATTTCCCAGGCTGTTGTGACTCAGGAATCTGCACTCACCAC |
| | | | ATCACCTGGTGAAACAGTCACACTCACTTGTCGCTCAAGTACTGGG |
| | | | GCTGTTACAACTAGTAACTATGCCAACTGGGTCCAAGAAAAACCAG |
| | | | ATCATTTATTCACTGGTCTAATAGGTGGTACCAACAACCGAGCTCC |
| | | | AGGTGTTCCTGCCAGATTCTCAGGCTCCCTGATTGGAGACAAGGCT |
| | | | GCCCTCACCATCACAGGGGCACAGACTGAGGATGAGGCAATATATC |
| | | | ACTGTGCTCTATGGTACAGCAACCTTTGGGTGTTCGGTGGAAGAAC |
| | | | CAAACTGACTGTCCTA |
| | Heavy (VH) | 30 | ATGGCTGTCCTGGTGCTGTTCCTCTGCCTGGTTGCATTTCCAAGCT |
| | | | GTGTCCTGTCCCAGGTGCAGCTGAAGGAGTCAGGACCTGGCCTGGT |
| | | | GGCGCCCTCACAGAGCCTGTCCATCACTTGCACTGTCTCTGGGTTT |
| | | | TCATTAACCAGCTATGGTGTACACTGGGTTCGCCAGCCTCCAGGAA |
| | | | AGGGTCTGGAGTGGCTGGGAGTAATATGGGCTGGTGGAGTCACACA |
| | | | TTATAATTCGGCTCTCATGTCCAGACTGAACATCAGCAAAGACAAC |
| | | | TCCCAGAGCCAAGTTTTCTTAAAAATGAACAGTCTACGAACTGATG |
| | | | ACACAGCCATGTACTACTGTGCCAGAGGTGGGGCATTACTTCGGTC |
| | | | TCACTTTGACTACTGGGGCCAAGGCACCACTCTCACAGTCTCCTCA |
| 908.9G5 | Light (VL) | 31 | ATGAGTGTGCCCACTCAGGTCCTGGGGTTGCTGCTGCTGTGGCTTA |
| | | | CAGGTGCCAGATGTGACATCCAGATGACTCAGTCTCCAGCCTCCCT |
| | | | ATCTGCATCTGTGGGAGAAACTGTCACCATCACATGTCGAGCAAGT |
| | | | GAGAATATTTTCATTTATTTAGCATGGTATCAGCAAAAACAGGGAA |
| | | | AATCTCCTCAGCTCCTGGTCTATAATGCAAAGACCTTAGCAGAAGG |
| | | | TGTGCCATCAAGGTTCAGTGGCAGTGGATCAGGCACACAGTTTTCT |
| | | | CTGAAGATCAACAGCCTTCTGCCTGAAGATTTTGGGAGTTATTACT |
| | | | GTCAACATCATTATGGTATTCCATTCACGTTCGGCTCGGGGACAAA |
| | | | GTTGGAAATAGAA |
| | Heavy (VH) | 32 | ATGAAATGCAGCTGGGTTATCTTCTTCCTGATGGCAGTGGTTACAG |
| | | | GGGTCAATTCAGAGGTTCAGCTGCAGCAGTCTGGGGCAGAGCTTGT |
| | | | GAAGCCAGGGGCCTCAGTCAAGTTGTCCTGCACAGTTTCTGGTTTC |
| | | | AACATTACAGACACCTATATACACTGGGTGAAGCAGAGGCCTGAAA |
| | | | AGGGCTTGGAGTGGATTGGAAGGATTGATCCTGCGAATGGTAATAC |
| | | | TAAATATGACCCGAAGTTACAGGGCAAGGCCACTATAACAGCAGAC |
| | | | ACATCCTCCAACACAGCCTACCTGCAGCTCAGCAGCCTGACATCTG |
| | | | AGGACACTGCCGTCTATTACTGTGCTAGAGATGGTACCTACGTTTA |
| | | | CTATGCTATGGACTACTGGGGTCAAGGAACCTCAGTCACCGTCTCC |
| | | | TCA |
| 903.8D8 | Light (VL) | 44 | ATGGATTTTCAAGTGCAGATTTTCAGCTTCCTGCTAATGAGTGCCT |
| | | | CAGTCATAATGTCCAGGGGACAAATTGTTCTCACCCAGTCTCCAGC |
| | | | ACTCATGTCTGCATCTCCAGGGGAGAAGGTCACCATGACCTGCAGT |
| | | | GCCAGCTCAAGTGTAAGTTACATGTACTGGTACCAGCAGAAGCCAA |
| | | | GATCCTCCCCCAAACCCTGGATTTATCTCACATCCAACCTGGCTTC |
| | | | TGGAGTCCCTGCTCGCTTCAGTGGCAGTGGGTCTGGGACCTCTTAC |
| | | | TCTCTCACAATCAGCAGCATGGAGGCTGAAGATGCTGCCACTTATT |
| | | | ACTGCCAGCAGTGGAGTAGTAACCCACTCACGTTCGGTGCTGGGAC |
| | | | CAAGCTGGAGCTGAAA |
| | Heavy (VH) | 45 | ATGATGGTGTTAAGTCTTCTGTACCTGTTGACAGCCCTTCCGGGTA |
| | | | TCCTGTCAGACGTGCAGCTTCAGGATCAGGACCTAGCCTCGTGAAA |
| | | | CCTTCTCAGACTCTGTCCCTCACCTGTTCTGTCACTGGCGACTCCA |
| | | | TCACAGTGGTTACTGGAACTGGATCCGGAAATTCCCAGGGAATAAA |
| | | | CTTGAGTACATGGGGTACATAAGCTACGTGGTAGCACTTACTACAA |
| | | | TCCATCTCTCAAAAGTCGAATCTCCATCACTCGAGACACATCCAAG |
| | | | AACCATACTACCTGCAGTTGAATTCTGTGACTACTGAGGACACAGC |
| | | | CACATATTACTGTGCAAGATGGGGGCATGTTACTACGGTAGTAGCT |
| | | | ACTGGTACTTCGATGTCTGGGGCGCAGGGACCACGGTCACCGTCTC |
| | | | CTCA |
| 908.11A2 | Light (VL) | 46 | ATGGTATCCACACCTCAGTTCCTTGTATTTTTGCTTTTCTGGATTC |
| | | | CAGCCTCCAGAAGTGACATCTTGCTGACTCAGTCTCCAGCCATCCT |
| | | | GTCTGTGAGTCCAGGAGACAGAGTCAGTTTCTCCTGCAGGGCCAGT |
| | | | CAGACCATTGGCACAAACATACACTGGTATCACCAAACAACAAATG |
| | | | GTTCTCCAAGGCTTCTCATTAAGTATGCTTCTGAGTCTATCTCTGG |
| | | | GATCCCTTCCAGGTTTAGTGGCAGTGGATCAGGGACAGATTTTACT |
| | | | CTTACCATCATCAGTGTGGAGTCTGAAGATATTGCAGATTATTACT |
| | | | GTCAACAAAGTAATAGCTGGCCGCTCACGTTCGGTGCTGGGACCAA |
| | | | GCTGGAGCTGAAA |
| | Heavy (VH) | 47 | ATGGGATGGAGCTGTATCATCCTCTTCTTGGTAGCAACAGCTACAG |
| | | | GTGTCCACTCCCAGGTCCAACTGCAGCAGCCTGGGGCTGAGCTGGT |
| | | | GAGGCCTGGGGCTTCAGTGAACCTGTCCTGCAAGGCTTCTGGCTAC |
| | | | ACCTTCACCAACTACTGGATAAACTGGGTGAAGCAGAGGCCTGGAC |
| | | | AAGGCCTTGAGTGGATCGGAAATATTCATCCTTATGATAGTTATAC |
| | | | TCACTACAATCAAAAGTTCAAGGGCAAGGCCACATTGACTGTAGAC |
| | | | AAATCCTCCAGCACGGCCTACATGCAGCTCAGCAGCCCGACATCTG |
| | | | AGGACTCTGCAGTCTATTTCTGTACAAGGAGGGGGGTACGACGGGA |
| | | | CGACTACTACTTTGACTACTGGGGCCAAGGCACCACTCTCACAGTC |
| | | | TCCTCA |

### Example 6: Specificity Test

The inventors tested the specificity of the antibodies of the invention by measuring the interaction of said antibodies with IL-1α and IL-1β, with IL-1R1 soluble form, IL-1R11 soluble form and co-receptor IL-1RAcP soluble form.

The experiments are carried out by sandwich capture. A first antibody is fixed on a support; the molecule to be tested is then added to allow an interaction, then a second biotinylated antibody is added. After washing, the fixed antibody-tested molecule-biotinylated antibody interaction is measured by adding steptavidin-HRP (horseradish peroxidase), then the revelation is done by measuring the luminescence at 450 nm in the presence of chromogenic substrate 3,3′,5,5′-tetramethylbenzidine (TMB).

The capture antibodies are fixed on the support at the rate of 1 μg/well.

The recombinant proteins to be tested are incubated for 2 h at ambient temperature on the fixed antibodies at the rate of 1 ng/mL of recombinant protein over 7 half-fold dilutions. The recombinant proteins are:
IL-1RA (R&D Systems, ref. 280-RA-050)
IL-1 (R&D Systems, ref. 200-LA-002/CF)α
IL-1 (R&D Systems, ref. 201-LB-005/CF)β
IL-1R1 soluble form (R&D Systems, ref. 269-21R-100/CF)
IL-1R11 soluble form (R&D Systems, ref. 263-2R-050/CF)
IL-1RAcP soluble form (R&D Systems, ref. 9176-CP-100)

This is followed by incubation for 1 hour at ambient temperature of the biotinylated revealing antibodies.

Several pairs of antibodies are used:
fixed 908.9G5/biotinylated 908.9E11
fixed 908.10G3/biotinylated 908.9E11
fixed 908.9G5/biotinylated 908.10E12
fixed 908.10G3/biotinylated 908.10E12, and
fixed 908.10G3/biotinylated 908.11H10.

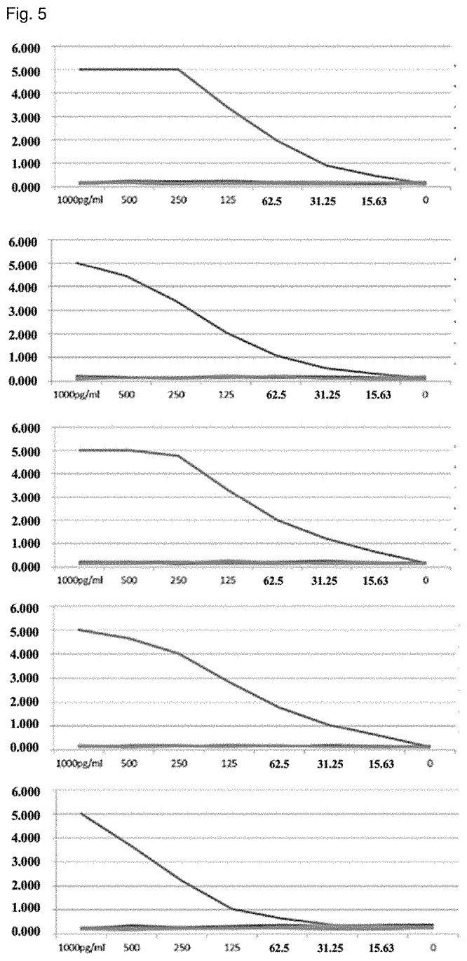

The results for each pair are shown in FIG. 5.

As the 5 curves show, whatever the pair of antibodies tested, no cross-reactivity is observed with IL-1α, IL-1β, the IL-1RI receptor, the IL-1RII receptor or the IL-1RAcP co-receptor.

### Example 7: Recognition of the Natural IL-1RA Protein

In order to ensure that the antibodies of the invention are capable not only of recognizing recombinant IL-1RA, but also native IL-1RA produced by human cells, the inventors took advantage of the properties of the THP-1 cell line to secrete soluble IL-1RA under stimulation with phorbol 12-myristate 13-acetate (PMA).

Thus, 5·10⁵ cells per mL of THP1 cells were cultured with 100 nM of PMA. The culture supernatants (SN) were harvested at 72 h and then tested on different pairs of antibodies.

The experimental conditions are as follows: Test conditions
Fixing: the capture antibodies are fixed at a rate of 1 μg/well
Incubation for 2 hours at room temperature of the recombinant IL-1RA range 1 ng/mL at ½ on 5 points or 10 μl of THP1+/− PMA cell supernatant
Incubation for 1 hour at room temperature of biotinylated revealing antibodies
Addition of streptavidin-HRP, then of TMB
Reading at 450 nm

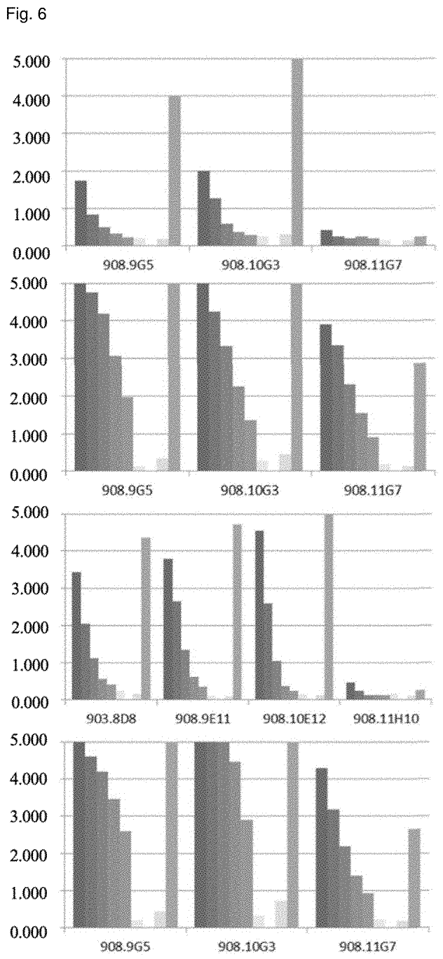

Representative results obtained are shown in FIG. 6.

Antibodies 908.11G7 and 908.11H10 are not very effective as capture antibodies and antibody 908.11A2 as biotinylated antibodies, so detection of soluble IL-1RA secreted by THP1 cells is not effective.

However, antibodies
908.9E11B6,
908.9G5B9, and
908.10E12C1,

are very effective and make it possible to recognize both the recombinant form and the natural form secreted by the THP1 cells.

### Example 8: IL-1RA Assay in Human Serum

The inventors also attempted to assay IL-1RA in serum from individuals, using antibody pairs according to the protocol used in Example 7, where the sera were diluted to 1/5 then to 1/2 on 4 points (100 μL per well). The capture antibodies are fixed at a rate of 1 μg/well.

The results are represented in the following tables:

| | Dilution | O.D. | [IL-1RA 1 pg/mL |
|---|---|---|---|
| Serum No. 1 | ⅕ | 1.435 | 64 |
| | 1/10 | 1.007 | 80 |
| | 1/20 | 0.686 | 88 |
| | 1/40 | 0.401 | 50 |
| Serum No. 2 | ⅕ | 1.858 | 87 |
| | 1/10 | 0.908 | 69 |
| | 1/20 | 0.613 | 72 |
| | 1/40 | 0.331 | 19 |
| Serum No. 3 | ⅕ | 0.571 | 16 |
| | 1/10 | 0.381 | 10 |
| | 1/20 | 0.431 | 32 |
| | 1/40 | 0.381 | 41 |
| Serum No. 4 | ⅕ | 1.328 | 58 |
| | 1/10 | 0.709 | 47 |
| | 1/20 | 0.46 | 38 |
| | 1/40 | 0.368 | 35 |

fixed 908.9G5/biotinylated 908.9E11

TABLE 4

| | Dilution | O.D. | [IL-1RA 1 pg/mL |
|---|---|---|---|
| Serum No. 1 | ⅕ | 1.192 | 107 |
| | 1/10 | 0.869 | 141 |
| | 1/20 | 0.606 | 163 |
| | 1/40 | 0.462 | 196 |
| Serum No. 2 | ⅕ | 2.048 | 204 |
| | 1/10 | 0.784 | 122 |
| | 1/20 | 0.546 | 136 |
| | 1/40 | 0.433 | 170 |
| Serum No. 3 | ⅕ | 0.626 | 43 |
| | 1/10 | 0.445 | 45 |
| | 1/20 | 0.341 | 43 |
| | 1/40 | 0.303 | 52 |
| Serum No. 4 | ⅕ | 1.171 | 105 |
| | 1/10 | 0.699 | 103 |
| | 1/20 | 0.426 | 82 |
| | 1/40 | 0.325 | 72 |

fixed 908.10G3/biotinylated 908.9E11

TABLE 5

| | Dilution | O.D. | [IL-1RA 1 pg/mL |
|---|---|---|---|
| Serum No. 1 | ⅕ | 1.109 | 50 |
| | 1/10 | 0.652 | 49 |
| | 1/20 | 0.472 | 59 |
| | 1/40 | 0.456 | 111 |
| Serum No. 2 | ⅕ | 1.41 | 66 |
| | 1/10 | 0.637 | 48 |
| | 1/20 | 0.439 | 52 |
| | 1/40 | 0.329 | 55 |
| Serum No. 3 | ⅕ | 0.46 | 14 |
| | 1/10 | 0.364 | 18 |
| | 1/20 | 0.299 | 21 |
| | 1/40 | 0.288 | 37 |
| Serum No. 4 | ⅕ | 1.029 | 45 |
| | 1/10 | 0.535 | 36 |
| | 1/20 | 0.419 | 47 |
| | 1/40 | 0.265 | 27 |

coated 908.9G5/biotinylated 908.10E12

TABLE 6

| | Dilution | O.D. | [IL-1RAIpg/mL |
|---|---|---|---|
| Serum No. 1 | ⅕ | 1.65 | 95 |
| | 1/10 | 1.057 | 107 |
| | 1/20 | 0.647 | 97 |
| | 1/40 | 0.54 | 134 |
| Serum No. 2 | ⅕ | 2.548 | 159 |
| | 1/10 | 1.031 | 103 |
| | 1/20 | 0.716 | 117 |
| | 1/40 | 0.366 | 35 |
| Serum No. 3 | ⅕ | 0.785 | 34 |
| | 1/10 | 0.429 | 18 |
| | 1/20 | 0.337 | 9 |
| | 1/40 | 0.393 | 50 |
| Serum No. 4 | ⅕ | 1.513 | 86 |
| | 1/10 | 0.775 | 67 |
| | 1/20 | 0.471 | 47 |
| | 1/40 | 0.371 | 38 |

coated 908.10G3/biotinylated 908.10E12

These results show that the selected pairs do not assay IL-1 RA containing in human serum in an equivalent way:
The two pairs fixed 908.10G3/biotinylated 908.9E11 and fixed 908.10G3/biotinylated 908.10E12 give concentrations that come closest to those described in the bibliography (of the order of 100 μg/mL, depending on the sera tested), and
The two coated 908.9G5/biotinylated 908.9E11 and coated 908.9G5/biotinylated 908.10E12 pairs assay lower serum IL-1RA (of the order of 50 ng/mL).

The two fixed 908.10G3/biotinylated 908.9E11 and fixed 908.10G3/biotinylated 908.10E12 pairs seem best suited for assaying serum IL-1RA.

### Example 9: “Spiking” Test

This test is based on a so-called “spiking” test with a calculation of the percentage of recovery, ratio of the quantity of protein assayed to the quantity of protein expected.

A test to assess the interaction of IL-RA with IL-1α, IL-1β, IL-1R1 soluble form, IL-1RII soluble form or IL-1RAcP soluble form was carried out on previously selected pairs, namely:
fixed 908.9G5/biotinylated 908.9E11,
fixed 908.10G3/biotinylated 908.9E11,
fixed 908.9G5/biotinylated 908.10E12, and
fixed 908.10G3/biotinylated 908.10E12.

The results obtained are as follows:

1—Fixed 908.9G5/biotinylated 908.9E11 pair
capture antibodies: 1 μg/well
IL-RA range 50 μg/mL at ¼ on 4 points diluted in a solution at 500 μg/mL
IL-1α (R&D Systems, ref. 200-LA-002/CF),
IL-1β (R&D Systems, ref. 201-LB-005/CF),
IL-1RI soluble form (R&D Systems, ref. 269-21R-100/CF),
IL-1RII soluble form (R&D Systems, ref. 263-2R-050/CF)
or IL-1RAcP soluble form (R&D Systems, ref. 9176-CP-100)

The labels and the incubation times, as well as the revelation, are the same as those described in the previous example.

The results are confined in the following Table 7:

TABLE 7

| IL-1RA | IL-1α | [IL-1RA assayed after spiking] pg/mL | | | |
|---|---|---|---|---|---|
| “spiked” | | 51 | 23 | 12 | 5 |
| with | | Recovery % | | | |
| | | 102 | 92 | 96 | 78 |
| | IL-1β | [IL-1RA assayed after spiking] pg/mL | | | |
| | | 52 | 19 | 8 | 2 |
| | | Recovery % | | | |
| | | 104 | 78 | 62 | 37 |
| | IL-1RI | [IL-1RA assayed after spiking] pg/mL | | | |
| | soluble | 46 | 15 | 8 | 4 |
| | form | Recovery % | | | |
| | | 91 | 62 | 60 | 61 |
| | IL-1RII | [IL-1RA assayed after spiking] pg/mL | | | |
| | soluble | 43 | 18 | 7 | 2 |
| | form | Recovery % | | | |
| | | 85 | 73 | 55 | 34 |
| | IL-1RAcP | [IL-1RA assayed after spiking] pg/mL | | | |
| | soluble | 45 | 16 | 7 | 3 |
| | form | Recovery % | | | |
| | | 90 | 65 | 57 | 47 |

2—Fixed 908.10G3/biotinylated 908.9E11 pair
capture antibodies: 1 μg/well
IL-RA range 100 μg/mL at ¼ on 4 points diluted in a 1 ng/mL solution
IL-1α (R&D Systems, ref. 200-LA-002/CF),
IL-1β (R&D Systems, ref. 201-LB-005/CF),
IL-1R1 soluble form (R&D Systems, ref. 269-21R-100/CF),
IL-1R11 soluble form (R&D Systems, ref. 263-2R-050/CF)
or IL-1RAcP soluble form (R&D Systems, ref. 9176-CP-100)

The results are confined in the following Table 8:

TABLE 8

| IL-1RA | IL-1α | [IL-1RA assayed after spiking] pg/mL | | | |
|---|---|---|---|---|---|
| “spiked” | | 85 | 37 | 21 | 13 |
| with | | Recovery % | | | |
| | | 85 | 74 | 86 | 107 |
| | IL-1β | [IL-1RA assayed after spiking] pg/mL | | | |
| | | 89 | 37 | 22 | 12 |
| | | Recovery % | | | |
| | | 89 | 75 | 86 | 98 |
| | IL-1RI | [IL-1RA assayed after spiking] pg/mL | | | |
| | soluble | 63 | 24 | 15 | 10 |
| | form | Recovery % | | | |
| | | 63 | 48 | 58 | 84 |
| | IL-1RII | [IL-1RA assayed after spiking] pg/mL | | | |
| | soluble | 83 | 40 | 19 | 12 |
| | form | Recovery % | | | |
| | | 83 | 80 | 75 | 92 |
| | IL-1RAcP | [IL-1RA assayed after spiking] pg/mL | | | |
| | soluble | 84 | 31 | 17 | 11 |
| | form | Recovery % | | | |
| | | 84 | 62 | 69 | 91 |

3—Fixed 908.9G5/biotinylated 908.10E12 pair
capture antibodies: 1 μg/well
IL-1RA range 50 μg/mL at ½ on 4 points diluted in a solution at 500 μg/mL
IL-1α (R&D Systems, ref. 200-LA-002/CF),
IL-1β (R&D Systems, ref. 201-LB-005/CF),
IL-1R1 soluble form (R&D Systems, ref. 269-21R-100/CF),
IL-1R11 soluble form (R&D Systems, ref. 263-2R-050/CF)
or IL-1RAcP soluble form (R&D Systems, ref. 9176-CP-100)

The results are confined in the following Table 8:

TABLE 9

| IL-1RA | IL-1α | [IL-1RA assayed after spiking] pg/mL | | | |
|---|---|---|---|---|---|
| “spiked” | | 46 | 19 | 10 | 5 |
| with | | Recovery % | | | |
| | | 93 | 75 | 77 | 77 |
| | IL-1β | [IL-1RA assayed after spiking] pg/mL | | | |
| | | 50 | 19 | 10 | 6 |
| | | Recovery % | | | |
| | | 100 | 78 | 82 | 99 |
| | IL-1RI | [IL-1RA assayed after spiking] pg/mL | | | |
| | soluble | 43 | 19 | 9 | 4 |
| | form | Recovery % | | | |
| | | 86 | 75 | 72 | 68 |
| | IL-1RII | [IL-1RA assayed after spiking] pg/mL | | | |
| | soluble | 49 | 18 | 10 | 5 |
| | form | Recovery % | | | |
| | | 98 | 73 | 82 | 88 |
| | IL-1RAcP | [IL-1RA assayed after spiking] pg/mL | | | |
| | soluble | 44 | 20 | 10 | 5 |
| | form | Recovery % | | | |
| | | 87 | 79 | 80 | 87 |

4—Fixed 908.10G3/biotinylated 908.10E12
capture antibodies: 1 μg/well
IL-RA range 100 μg/mL at ½ on 4 points diluted in a solution at 500 μg/mL
IL-1α (R&D Systems, ref. 200-LA-002/CF),
IL-1β (R&D Systems, ref. 201-LB-005/CF),
IL-1R1 soluble form (R&D Systems, ref. 269-21R-100/CF),
IL-1R11 soluble form (R&D Systems, ref. 263-2R-050/CF)
or IL-1RAcP soluble form (R&D Systems, ref. 9176-CP-100)

The results are confined in the following Table 8:

TABLE 10

| IL-1RA | IL-1α | [IL-1RA assayed after spiking] pg/mL | | | |
|---|---|---|---|---|---|
| “spiked” | | 46 | 21 | 11 | 6 |
| with | | Recovery % | | | |
| | | 92 | 83 | 87 | 97 |
| | IL-1β | [IL-1RA assayed after spiking] pg/mL | | | |
| | | 44 | 21 | 12 | 6 |
| | | Recovery % | | | |
| | | 89 | 84 | 95 | 98 |
| | IL-1RI | [IL-1RA assayed after spiking] pg/mL | | | |
| | soluble | 40 | 19 | 10 | 6 |
| | form | Recovery % | | | |
| | | 80 | 77 | 77 | 99 |
| | IL-1RII | [IL-1RA assayed after spiking] pg/mL | | | |
| | soluble | 44 | 21 | 12 | 6 |
| | form | Recovery % | | | |
| | | 89 | 84 | 95 | 93 |
| | IL-1RAcP | [IL-1RA assayed after spiking] pg/mL | | | |
| | soluble | 45 | 19 | 11 | 8 |
| | form | Recovery % | | | |
| | | 90 | 78 | 92 | 122 |

A recovery percentage is considered good for values between 80 and 120%, i.e. a variation of 20% between the quantity measured and the quantity theoretically added. In general, the lower the quantity of protein to be detected, the more the variation in the recovery percentage tends to increase beyond 20%.

In this test, the interactions between proteins are evaluated at a ratio of 10 at the first point, but this ratio is almost 100 for the fourth point, which makes good quantification even more difficult at low concentrations.

The recovery percentages found in these tests are good or very good

This means that the evaluated proteins do not interfere with the assay of IL-1RA, at least for an assay carried out with the first four pairs.

### Example 10: Epitope Mapping

The epitope mapping of the 3 blocking antibodies selected in examples 2, 3 and 4 and of two non-blocking antibodies (903.8D8B1 and 908.11A2H9) was evaluated using the MAbTope software (MabSilico).

This software allows the construction of 3D structural models of the target end of antibodies from models used to model the heavy chain domain (VH) and the light chain domain (VL) as well as the relative orientation between the VH and VL domains.

The modeling data used for the 5 antibodies are summarized in the following table:

TABLE 11

| | | | Orientation based |
|---|---|---|---|
| Antibody | VH Model | VL Model | on |
| 908.9E11B6 | PBD: 3vfg | PBD: 7jti | PBD: 3vfg |
| 908.10E12C1 | PBD: 1a6t | PBD: 6bt3 | PBD: 1a6t |
| 908.9G5B9 | PBD: 5hdb | PBD: 1kb5 | PBD: 5hdb |
| 903.8D8B1 | PBD: 1dqd | PBD: 1sy6 | PBD: 1dqd |
| 908.11A2H9 | PBD: 1iqw | PBD: 4krp | PBD: 1iqw |

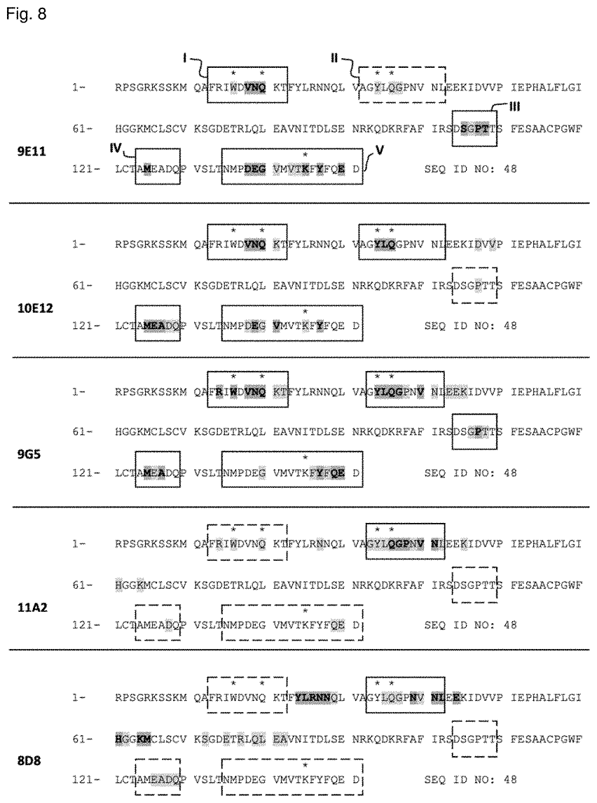

The results obtained are presented in FIG. 8.

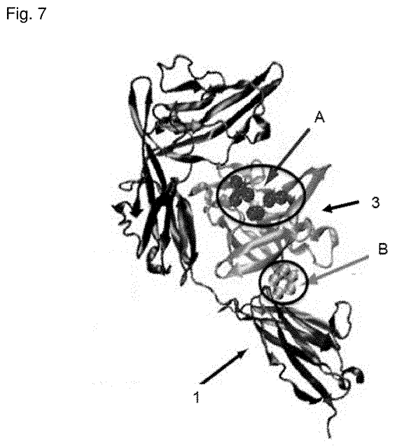

In FIG. 7, a three-dimensional representation of the binding of IL-1RA with the IL-1 receptor can be seen. This binding is carried out through two sites carried by IL-1RA: a first binding site A corresponds to the domain centered on Lysine in position 145 (K145) of amino acid sequence SEQ ID NO: 48 and a second binding site B formed by W16, Q20, Y34 and Q36 of amino acid sequence SEQ ID NO: 48. To facilitate reading, the amino acids in question have been annotated in FIG. 8 with a star over each IL-1RA amino acid sequence shown.

From the results obtained, 5 common recognition sequences (modules numbered from I to V) were able to be determined. Regarding the mapping of the 903.8D8B1 antibody, particular sequences (unboxed) can also be considered (SEQ ID NO: 41, SEQ ID NO: 42 and SEQ ID NO: 43) to define the epitope.

In FIG. 8, it is noted that the epitopes of the blocking antibodies (9E11, 10E12 and 9G5) all have sequences I and V of IL-1RA. These sequences partially cover binding site B and completely cover binding site A of IL-1RA on the receptor. Conversely, these two sequences are not recognized by either of the two non-blocking antibodies (11A2 and 8D8). In view of the results, sequences I and V appear to be essential for obtaining a blocking function by the antibodies for the binding of IL-1RA to the receptor.

It is also noted that sequence IV of IL-1RA is recognized by all of the blocking antibodies and none of the non-blocking antibodies.

Sequence II, corresponding to the third binding site of IL-1RA, is recognized by two (908.10E12C1 and 908.9G5B9) of the three blocking antibodies and by the two non-blocking antibodies. The results obtained by this evaluation of the epitopes are consistent with the blocking and non-blocking functions of the antibodies.

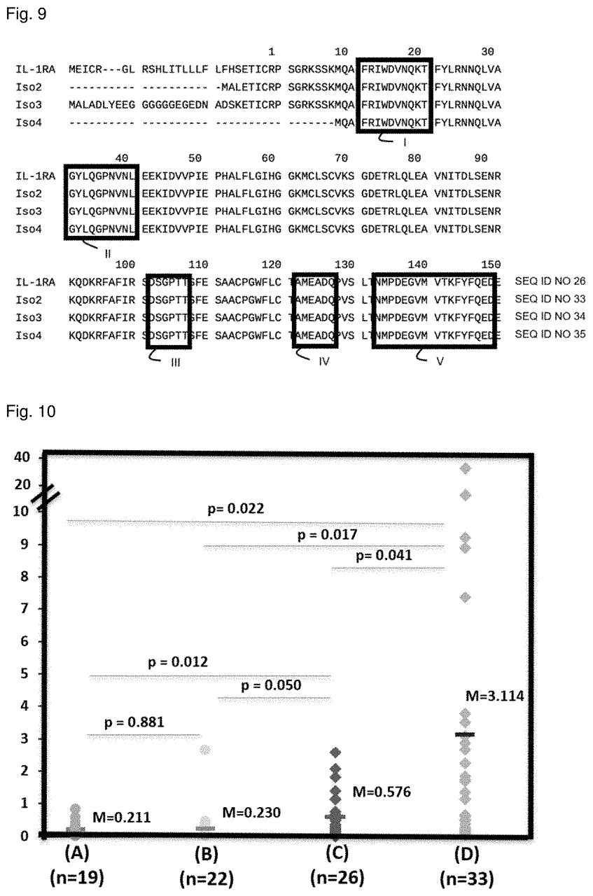

FIG. 9 shows that sequences I to V are all present on the different isoforms of IL-1RA, which confirms that the antibodies according to the invention are capable of recognizing all the isoforms of IL-1RA.

### Example 11: Diagnosis of the Chronic Form of Lyme Disease

In this example, the inventors have shown that an ELISA using the anti-IL-1RA monoclonal antibodies according to the invention advantageously makes it possible to obtain diagnostic results in patients suffering from a chronic form of Lyme disease. In addition, the inventors have demonstrated that using the antibodies according to the invention in the context of determining the ratio of IL-1RA/interferon-γ concentrations makes it possible to be discriminating with respect to several groups of patients affected by Lyme disease at different stages. To this end, the 908.10G3 antibody was used as the fixing antibody, and the 908.9E11 antibody conjugated with biotin was used as the tracer, as described in detail in the aforementioned table no. 4

The patients tested were separated into 4 groups: Group A (healthy control); Group B (patients with an acute form of Lyme disease—without immunosuppression); Group C (patients with an acute form of Lyme disease who relapsed from the initial treatment); Group D (patients developing remote Lyme pathology in a chronic form).

The results are shown in FIG. 10.

These results show that the ratio of IL-1RA/interferon-γ concentrations is identical in healthy people (Group A) and patients who have just been bitten by a tick infected with Borrelia (Group B) (p=0.881). The results also show that the ratio of IL-1RA/interferon-γ concentrations is significantly different between group A and group C or D (p=0.012; p=0.022), between group B and group C or D (p=0.050; p=0.017) as well as between group C and group D (p=0.041).

It can be concluded from these results that the antibodies according to the invention can be used for diagnosis of the complex forms of Lyme disease and differentiation in patients of the different stages of the disease. Furthermore, these results strengthen the diagnostic link between the amounts of IL-1RA and IFN differentially synthesized by healthy people or people developing an acute response with cutaneous inflammation, versus people who show immunosuppression during a relapse to the initial treatment or late and chronic forms of the disease.

### Example 12: Treatment of the Chronic Form of Lyme Disease

The treatment of the chronic form of Lyme disease was carried out on a mouse model and a rhesus macaque model. The objective was to analyze the effect of blocking IL-1RA in resolving late Lyme disease in these animals, and the possibility of reversing the development of the disease by treating them with antibodies neutralizing the IL-1RA associated with antibiotics. Another aspect of the analysis was also to study the effect of exogenous IL-1RA intake during the disease, and to analyze the impact on the increase in the pathological response.

To this end, the inventors used IL-1RA neutralizing antibodies according to the invention, isotype control IgG antibodies, and mouse or macaque IL-1RA. Response tracking essentially focused on the analysis of the osteo-articular component of the disease.

The mice/macaques were infected with the N40 strain of B. burgdorferi (50,000 bacteria) as previously described for the mouse model (Jie Feng et al., Discov Med 27(148):125-138, March 2019).

For each experiment (mice and macaques), 13 groups of 6 individuals each were distributed as follows:

(1) Doxycycline treatment only

(2) infection only

(3) infection+doxycycline

(4) doxycycline+anti-IL-1RA antibodies

(5) infection+anti-IL-1RA antibodies

(6) infection+doxycycline+anti-IL-1RA antibodies

(7) infection control+doxycycline+IgG

(8) doxycycline+IgG control

(9) infection control+IgG

(10) infection control+doxycycline+IgG

(11) doxycycline+IL-1RA

(12) infection+IL-1RA

(13) infection+doxycycline+IL-1RA

For the mice, the injections of anti-IL-1RA antibodies and the IgG control consisted of intraperitoneal (IP) injections (90 micrograms) once a week, starting on day 21 (days 21, 28, 35). Doxycycline treatment consisted of oral administration twice a day for 21 days. IL-1RA injections consisted of IP injections (2 micrograms/injection) every 3 days, starting on day 21 (days 21, 24, 27, 30, 33, 36 and 39). Treatments began on day 21 post-infection. An autopsy was early endpoint at day 49 (n=3), or late endpoint at day 90 (n=3). Joint swelling was measured with a caliper up to 6 times between days 14 and 28 of infection. Blood was collected on day 0, every 2 weeks until autopsy, and on days 1, 3, and 5 during antibody and/or doxycycline treatment.

The treatment protocol for rhesus macaques was the same as for mice, notwithstanding that the dosages and reagents were adapted to the size and species of macaques.

Furthermore, the inventors have carried out preclinical tests on humans using an IL-1RA neutralizing antibody according to the invention to prevent the inflammation of the Borrelia burgdorferi infection. A preclinical test of the impact of the cytokine IL-1RA on the increase in the pathological response was also carried out.

1-14. (canceled)

15. An isolated monoclonal antibody against an interleukin 1 receptor antagonist, or IL-1RA, recognizing the peptide of amino acid sequence SEQ ID NO: 25, said antibody inhibiting the interaction between the interleukin-1 receptor antagonist and the interleukin-1 receptor, and having an affinity for the interleukin-1 receptor antagonist of less than 10⁻⁸ M, measured by the OCTET technique,
or one of the derivative compounds or functional fragments of said antibody.

16. The antibody according to claim 15, recognizing a conformational or linear epitope of IL-1RA, said conformational or linear epitope comprising at least amino acid sequences SEQ ID NO: 36 and SEQ ID NO: 40 belonging to amino acid sequence SEQ ID NO: 25.

17. The antibody according to claim 16, wherein said conformational or linear epitope comprises at least sequences SEQ ID NO: 36, SEQ ID NO: 39 and SEQ ID NO: 40 belonging to amino acid sequence SEQ ID NO: 25.

18. The antibody according to claim 16, wherein said conformational or linear epitope further comprises amino acid sequences SEQ ID NO: 37 belonging to amino acid sequence SEQ ID NO: 25.

19. The antibody according to claim 15, comprising a light chain comprising at least one CDR of the group consisting of CDR-L1, CDR-L2 and CDR-L3, wherein:
CDR-L1 comprises one of the sequences SEQ ID NO: 1 (KSSQSLLNSSNQKNYLA), SEQ ID NO: 4 and SEQ ID NO: 7,
CDR-L2 comprises one of the sequences SEQ ID NO: 2, SEQ ID NO: 5, and SEQ ID NO: 8,
CDR-L3 comprises one of the sequences SEQ ID NO: 3, SEQ ID NO: 6 and SEQ ID NO: 9,
or one of its derivative compounds or functional fragments.

20. The antibody according to claim 15, comprising a heavy chain comprising at least one CDR of the group consisting of CDR-H1, CDR-H2 and CDR-H3, where
CDR-H1 comprises one of the sequences SEQ ID NO: 10 (EYTMH), SEQ ID NO: 13 and SEQ ID NO: 16,
CDR-H2 comprises one of the sequences SEQ ID NO: 11, SEQ ID NO: 14 and SEQ ID NO: 17,
CDR-H3 comprises one of the sequences SEQ ID NO: 12, SEQ ID NO: 15 and SEQ ID NO: 18,
or one of its derivative compounds or functional fragments.

21. The antibody according to claim 15, said antibody comprising a light chain comprising:
the CDRs of sequences SEQ ID NO: 1 to 3, or
the CDRs of sequences SEQ ID NO: 4 to 6, or
the CDRs of sequences SEQ ID NO: 7 to 9.

22. The antibody according to claim 15, said antibody comprising a heavy chain comprising:
the CDRs of sequences SEQ ID NO: 10 to 12, or
the CDRs of sequences SEQ ID NO: 13 to 15, or
the CDRs of sequences SEQ ID NO: 16 to 18.

23. The antibody according to claim 15, said antibody comprising:
a variable part of the light chain comprising any one of the following sequences SEQ ID NO: 19 to 21, and
a variable part of the heavy chain comprising any one of the following sequences SEQ ID NO: 22 to 24.

24. A Nucleic acid molecule coding for a light chain of the monoclonal antibody as defined in claim 15.

25. A Nucleic acid molecule coding for a heavy chain of the monoclonal antibody as defined in claim 15.

26. A method for treating an inflammatory, infectious or autoimmune disease, the method comprising the administration to an individual in a need thereof of an effective amount of one of:
an isolated monoclonal antibody against the interleukin 1 receptor antagonist, or IL-1RA, recognizing the peptide of amino acid sequence SEQ ID NO: 25, wherein the antibody inhibits the interaction between the interleukin-1 receptor antagonist and the interleukin-1 receptor, and has an affinity for the interleukin-1 receptor antagonist of less than 10⁻⁸ M, measured by the OCTET technique, or a functional fragment thereof; and
a nucleic acid molecule coding for the isolated monoclonal antibody or the functional fragment thereof.

27. A method for the diagnosis of an inflammatory, infectious or autoimmune diseases, the method comprising
bringing a biological sample from an individual suspected of suffering from the inflammatory, infectious or autoimmune disease into contact with an isolated monoclonal antibody against an interleukin 1 receptor antagonist, or IL-1RA, recognizing the peptide of amino acid sequence SEQ ID NO: 25, said antibody inhibiting the interaction between the interleukin-1 receptor antagonist and the interleukin-1 receptor, and having an affinity for the interleukin-1 receptor antagonist of less than 10⁻⁸ M, measured by the OCTET technique,
detecting the presence or absence of, or the amount of, the IL-1RA protein in said sample, and
comparing the presence or absence of, or the amount of, the IL-1RA protein with the presence or absence of, or the amount of, the IL-1RA protein in at least one reference sample, whether it is a positive control or a negative control or both.

28. The antibody according to claim 15, said antibody comprising one of:
a variable part of the light chain of sequence SEQ ID NO: 19 and a variable part of the heavy chain of sequence SEQ ID NO: 22, or
a variable part of the light chain of sequence SEQ ID NO: 20 and a variable part of the heavy chain of sequence SEQ ID NO: 23, or
a variable part of the light chain of sequence SEQ ID NO: 21 and a variable part of the heavy chain of sequence SEQ ID NO: 24.

29. The method according to claim 26, wherein the inflammatory, infectious or autoimmune disease is a Lyme disease.

30. The method according to claim 26, wherein the inflammatory, infectious or autoimmune disease is a chronic disseminated form of Lyme disease.

31. The method according to claim 27, wherein the inflammatory, infectious or autoimmune disease is a Lyme disease.

32. The method according to claim 27, wherein the inflammatory, infectious or autoimmune disease is a chronic disseminated form of Lyme disease.
